(19) United States
(12) Patent Application Publication
(10) Pub. No.: US 2022/0333054 A1
(43) Pub. Date: Oct. 20, 2022

(54) METHODS OF PERFUSION CULTURING A MAMMALIAN CELL

(71) Applicant: Genzyme Corporation, Cambridge, MA (US)

(72) Inventors: Shawn L. Barrett, Norfolk, MA (US); Charles Budde, Arlington, MA (US); Daryl Powers, Weston, MA (US)

(21) Appl. No.: 17/721,003

(22) Filed: Apr. 14, 2022

Related U.S. Application Data
(60) Provisional application No. 63/174,900, filed on Apr. 14, 2021.

(51) Int. Cl. C12M 1/00; C12N 5/00

(52) U.S. Cl. CPC C12M 29/10; C12N 5/0018

(57) ABSTRACT

Provided herein are methods of perfusion culturing a mammalian cell that include: providing a vessel containing a mammalian cell disposed in a first liquid culture medium having an osmolality of about 270 mOsm/kg to about 380 mOsm/kg; incubating the mammalian cell for a period of time at about 32° C. to about 39° C.; and during the period of time, continuously or periodically removing a first volume of the first liquid culture medium and adding to the first liquid culture medium a second volume of a second liquid culture medium, wherein the first and second volumes are about equal and the osmolality of the first and second liquid culture medium in the vessel is maintained at about 270 mOsm/kg to about 380 mOsm/kg over the period of time.

### CROSS-REFERENCE TO RELATED APPLICATIONS

This application claims priority to U.S. Provisional Patent Application Ser. No. 63/174,900, filed Apr. 14, 2021; the entire contents of this application is herein incorporated by reference.

### TECHNICAL FIELD

This invention relates to methods of biotechnology and the manufacturing of recombinant proteins.

### BACKGROUND

Mammalian cells containing a nucleic acid that encodes a recombinant protein are often used to produce therapeutically or commercially important proteins. In the current environment of diverse product pipelines, biotechnology companies are increasingly driven to develop innovative solutions for highly flexible and cost-effective manufacturing of therapeutic protein drug substances.

### SUMMARY

The present invention is based, at least in part, on the discovery that methods of perfusion culturing a mammalian cell that include: providing a vessel containing a mammalian cell disposed in a first liquid culture medium having an osmolality of about 270 mOsm/kg to about 380 mOsm/kg; incubating the mammalian cell for a period of time at about 32° C. to about 39° C.; and during the period of time, continuously or periodically removing a first volume of the first liquid culture medium and adding to the first liquid culture medium a second volume of a second liquid culture medium, wherein the first and second volumes are about equal, and the osmolality of the first liquid culture medium and the osmolality of the second liquid culture medium in the vessel is maintained at about 270 mOsm/kg to about 380 mOsm/kg over the period of time, achieve an improvement in culture growth and health as compared to other perfusion cell culturing methods where the osmolality is not maintained at about 270 mOsm/kg to about 380 mOsm/kg over the period of time.

Provided herein are methods of perfusion culturing a mammalian cell that include: providing a vessel containing a mammalian cell disposed in a first liquid culture medium having an osmolality of about 270 mOsm/kg to about 380 mOsm/kg (e.g., any of the subranges therein); incubating the mammalian cell for a period of time at about 32° C. to about 39° C. (e.g., any of the subranges therein); and during the period of time, continuously or periodically removing a first volume of the first liquid culture medium and adding to the first liquid culture medium a second volume of a second liquid culture medium, wherein the first and second volumes are about equal and the osmolality of the first and second liquid culture medium in the vessel is maintained at about 270 mOsm/kg to about 380 mOsm/kg (e.g., any of the subranges therein) over the period of time.

In some embodiments, the first liquid culture medium has an osmolality of about 270 mOsm/kg to about 320 mOsm/kg. In some embodiments, the second liquid culture medium includes greater than 8 g/L of a poloxamer. In some embodiments, the second liquid culture medium includes greater than 10 g/L of a poloxamer. In some embodiments, the second liquid culture medium includes greater than 12 g/L of a poloxamer. In some embodiments of any of the methods described herein, the poloxamer is Pluronic F68. In some embodiments of any of the methods described herein, the second liquid culture medium has a pH of about 6.8 to about 7.1. In some embodiments of any of the methods described herein, the second liquid culture medium includes sodium chloride. In some embodiments of any of the methods described herein, the second liquid culture medium has an osmolality of about 800 mOsm/kg to about 2,500 mOsm/kg. In some embodiments of any of the methods described herein, the second liquid culture medium has an osmolality of about 1,200 mOsm/kg to about 1,800 mOsm/kg. In some embodiments of any of the methods described herein, the second volume of the second liquid culture medium added is increased over the period of time. In some embodiments of any of the methods described herein, the second volume of the second liquid culture medium added is increased based on the viable cell density over the period of time. In some embodiments of any of the methods described herein, maintaining the osmolality of the first and second liquid culture medium in the vessel includes adjusting the flow rate of the second liquid culture medium at some point during the period of time.

In some embodiments, the adjusting step includes increasing the flow rate of the second liquid culture medium at some point during the period of time.

In some embodiments of any of the methods described herein, the method includes adjusting one or both of the pH and the pCO₂ of the first and second liquid culture medium in the vessel at some point during the period of time.

In some embodiments, the adjusting step includes decreasing the flow rate of the second liquid culture medium at some point during the period of time.

In some embodiments of any of the methods described herein, the method further includes increasing the pCO₂ and increasing the pH of the first and the second liquid culture medium in the vessel over the period of time.

In some embodiments, the pCO₂ and the pH of the first and the second liquid culture medium in the vessel are increased based on the viable cell density over the period of time.

In some embodiments of any of the methods described herein, a viable cell density of greater than 60×10⁶ cells/mL in the first and second liquid culture medium is achieved during the period of time. In some embodiments, a viable cell density of greater than 100×10⁶ cells/mL in the first and second liquid culture medium is achieved during the period of time. In some embodiments, a viable cell density of greater than 120×10⁶ cells/mL in the first and second liquid culture medium is achieved during the period of time.

In some embodiments of any of the methods described herein, the method further includes monitoring the osmolality of the first and second liquid culture medium in the vessel during the period of time.

In some embodiments of any of the methods described herein, the removing of the first volume of the first liquid culture medium, and the adding of the second volume of the second liquid culture medium during the period of time is performed simultaneously. In some embodiments of any of the methods described herein, the removing of the first volume of the first liquid culture medium, and the adding of the second volume of the second liquid culture medium during the period of time is performed continuously. In some embodiments of any of the methods described herein, the removing of the first volume of the first liquid culture medium, and the adding of the second volume of the second liquid culture medium over the period of time is performed periodically.

In some embodiments of any of the methods described herein, the method further includes periodically adding a third volume of an aqueous solution to the first and the second liquid culture medium in the vessel during the period of time, wherein the first volume and the sum of the second and third volumes are about equal and the osmolality of the first, second, and third liquid culture medium in the vessel is maintained at about 270 mOsm/kg to about 380 mOsm/kg over the period of time.

In some embodiments, the aqueous solution is a salt solution. In some embodiments, the salt solution is a sodium chloride solution or a potassium chloride solution.

In some embodiments of any of the methods described herein, the removing of the first volume of the first liquid culture medium, the adding of the second volume of the second liquid culture medium, and the adding of the third volume during the period of time is performed simultaneously. In some embodiments of any of the methods described herein, the removing of the first volume of the first liquid culture medium, the adding of the second volume of the second liquid culture medium, and the adding of the third volume during the period of time is performed continuously. In some embodiments of any of the methods described herein, the removing of the first volume of the first liquid culture medium, the adding of the second volume of the second liquid culture medium, and the adding of the third volume over the period of time is performed periodically.

In some embodiments of any of the methods described herein, the method further includes continuously or periodically adding to the first liquid culture medium a third volume of a third liquid culture medium, wherein the first volume and the sum of the second and third volumes are about equal and the osmolality of the first, second, and third liquid culture medium in the vessel is maintained at about 270 mOsm/mg to about 380 mOsm/kg over the period of time.

In some embodiments, the third liquid culture medium has an osmolality that is about 270 mOsm/kg to about 380 mOsm/kg. In some embodiments of any of the methods described herein, maintaining the osmolality of the first, second, and third liquid culture medium in the vessel includes adjusting the flow rate of the second liquid culture medium during the period of time. In some embodiments of any of the methods described herein, the adjusting step includes increasing the flow rate of the second liquid culture medium at some point during the period of time. In some embodiments of any of the methods described herein, the adjusting step includes decreasing the flow rate of the second liquid culture medium at some point during the period of time.

In some embodiments, the third liquid culture medium has an osmolality of about 10 mOsm/kg to about 270 mOsm/kg. In some embodiments, the third liquid culture medium has an osmolality of about 50 mOsm/kg to about 200 mOsm/kg.

In some embodiments of any of the methods described herein, maintaining the osmolality of the first, second, and third liquid culture medium in the vessel includes adjusting the flow rate of the third liquid culture medium during the period of time.

In some embodiments of any of the methods described herein, the adjusting step includes increasing the flow rate of the third liquid culture medium at some point during the period of time.

In some embodiments of any of the methods described herein, the adjusting step includes decreasing the flow rate of the third liquid culture medium at some point during the period of time.

In some embodiments of any of the methods described herein, the method further includes adjusting one or both of the pH and the pCO₂ of the first, second, and third liquid culture medium in the vessel at some point during the period of time. In some embodiments, the method includes increasing the pCO₂ and increasing the pH of the first, second, and third liquid culture medium in the vessel over the period of time. In some embodiments, the pCO₂ and the pH of the first, second, and third liquid culture medium in the vessel are increased based on the viable cell density over the period of time.

In some embodiments of any of the methods described herein, a viable cell density of greater than 60×10⁶ cells/mL in the first, second, and third liquid culture medium is achieved during the period of time. In some embodiments, a viable cell density of greater than 100×10⁶ cells/mL in the first, second, and third liquid culture medium is achieved during the period of time. In some embodiments, a viable cell density of greater than 120×10⁶ cells/mL in the first, second, and third liquid culture medium is achieved during the period of time.

In some embodiments of any of the methods described herein, the method further includes monitoring the osmolality of the first, second, and third liquid culture medium in the vessel during the period of time. In some embodiments of any of the methods described herein, the removing of the first volume of the first liquid culture medium, the adding of the second volume of the second liquid culture medium, and the adding of the third volume during the period of time is performed simultaneously. In some embodiments of any of the methods described herein, the removing of the first volume of the first liquid culture medium, the adding of the second volume of the second liquid culture medium, and the adding of the third volume during the period of time is performed continuously. In some embodiments of any of the methods described herein, the removing of the first volume of the first liquid culture medium, the adding of the second volume of the second liquid culture medium, and the adding of the third volume over the period of time is performed periodically.

In some embodiments of any of the methods described herein, the mammalian cell is a CHO cell.

In some embodiments of any of the methods described herein, the vessel is a bioreactor. In some embodiments, the bioreactor is a perfusion bioreactor.

Also provided herein are methods of producing a recombinant protein that include: providing a vessel containing a mammalian cell containing a nucleic acid encoding a recombinant protein disposed in a first liquid culture medium having an osmolality of about 270 mOsm/kg to about 380 mOsm/kg (e.g., any of the subranges therein); incubating the mammalian cell for a period of time at about 32° C. to about 39° C. (e.g., any of the subranges therein); and during the period of time, continuously or periodically removing a first volume of the first liquid culture medium and adding to the first liquid culture medium a second volume of a second liquid culture medium, wherein the first and second volumes are about equal and the osmolality of the first and second liquid culture medium in the vessel is maintained at about 270 mOsm/kg (e.g., any of the subranges therein) to about 380 mOsm/kg over the period of time; and recovering the recombinant protein from the mammalian cell or from the first and/or second liquid culture medium.

In some embodiments, the first liquid culture medium has an osmolality of about 270 mOsm/kg to about 320 mOsm/kg. In some embodiments, the second liquid culture medium includes greater than 8 g/L of a poloxamer. In some embodiments, the second liquid culture medium includes greater than 10 g/L of a poloxamer. In some embodiments, the second liquid culture medium includes greater than 12 g/L of a poloxamer. In some embodiments of any of the methods described herein, the poloxamer is Pluronic F68. In some embodiments of any of the methods described herein, the second liquid culture medium has a pH of about 6.8 to about 7.1. In some embodiments of any of the methods described herein, the second liquid culture medium includes sodium chloride.

In some embodiments of any of the methods described herein, the second liquid culture medium has an osmolality of about 800 mOsm/kg to about 2,500 mOsm/kg. In some embodiments, the second liquid culture medium has an osmolality of about 1,200 mOsm/kg to about 1,800 mOsm/kg. In some embodiments of any of the methods described herein, the second volume of the second liquid culture medium added is increased over the period of time. In some embodiments, the second volume of the second liquid culture medium added is increased based on the viable cell density over the period of time.

In some embodiments of any of the methods described herein, maintaining the osmolality of the first and second liquid culture medium in the vessel includes adjusting the flow rate of the second liquid culture medium at some point during the period of time. In some embodiments, the adjusting step includes increasing the flow rate of the second liquid culture medium at some point during the period of time. In some embodiments, the method further includes adjusting one or both of the pH and the pCO₂ of the first and second liquid culture medium in the vessel at some point during the period of time. In some embodiments, the adjusting step includes decreasing the flow rate of the second liquid culture medium at some point during the period of time.

In some embodiments of any of the methods described herein, the method further includes increasing the pCO₂ and increasing the pH of the first and the second liquid culture medium in the vessel over the period of time. In some embodiments, the pCO₂ and the pH of the first and the second liquid culture medium in the vessel are increased based on the viable cell density over the period of time.

In some embodiments of any of the methods described herein, a viable cell density of greater than 60×10⁶ cells/mL in the first and second liquid culture medium is achieved during the period of time. In some embodiments, a viable cell density of greater than 100×10⁶ cells/mL in the first and second liquid culture medium is achieved during the period of time. In some embodiments, a viable cell density of greater than 120×10⁶ cells/mL in the first and second liquid culture medium is achieved during the period of time.

In some embodiments of any of the methods described herein, the method further includes monitoring the osmolality of the first and second liquid culture medium in the vessel during the period of time. In some embodiments of any of the methods described herein, the removing of the first volume of the first liquid culture medium, and the adding of the second volume of the second liquid culture medium during the period of time is performed simultaneously. In some embodiments of any of the methods described herein, the removing of the first volume of the first liquid culture medium, and the adding of the second volume of the second liquid culture medium during the period of time is performed continuously. In some embodiments of any of the methods described herein, the removing of the first volume of the first liquid culture medium, and the adding of the second volume of the second liquid culture medium over the period of time is performed periodically.

In some embodiments of any of the methods described herein, the recombinant protein is secreted into the first and/or second liquid culture medium. In some embodiments, the recombinant protein is recovered from the first and/or second liquid culture medium.

In some embodiments of any of the methods described herein, the method further includes continuously or periodically adding a third volume of an aqueous solution to the first and the second liquid culture medium in the vessel during the period of time, wherein the first volume and the sum of the second and third volumes are about equal and the osmolality of the first, second, and third liquid culture medium in the vessel is maintained at about 270 mOsm/kg to about 380 mOsm/kg over the period of time. In some embodiments, the aqueous solution is a salt solution. In some embodiments, the salt solution is a sodium chloride solution or a potassium chloride solution.

In some embodiments of any of the methods described herein, the removing of the first volume of the first liquid culture medium, the adding of the second volume of the second liquid culture medium, and the adding of the third volume during the period of time is performed simultaneously. In some embodiments of any of the methods described herein, the removing of the first volume of the first liquid culture medium, the adding of the second volume of the second liquid culture medium, and the adding of the third volume during the period of time is performed continuously. In some embodiments of any of the methods described herein, the removing of the first volume of the first liquid culture medium, the adding of the second volume of the second liquid culture medium, and the adding of the third volume over the period of time is performed periodically.

In some embodiments of any of the methods described herein, the method further includes continuously or periodically adding to the first liquid culture medium a third volume of a third liquid culture medium, wherein the first volume and the sum of the second and third volumes are about equal and the osmolality of the first, second, and third liquid culture medium in the vessel is maintained at about 270 mOsm/kg to about 380 mOsm/kg over the period of time.

In some embodiments, the third liquid culture medium has an osmolality that is about 270 mOsm/kg to about 380 mOsm/kg. In some embodiments of any of the methods described herein, maintaining the osmolality of the first, second, and third liquid culture medium in the vessel includes adjusting the flow rate of the second liquid culture medium during the period of time. In some embodiments of any of the methods described herein, the adjusting step includes increasing the flow rate of the second liquid culture medium at some point during the period of time. In some embodiments of any of the methods described herein, the adjusting step includes decreasing the flow rate of the second liquid culture medium at some point during the period of time. In some embodiments, the third liquid culture medium has an osmolality of about 0 mOsm/kg to about 270 mOsm/kg. In some embodiments, the third liquid culture medium has an osmolality of about 50 mOsm/kg to about 200 mOsm/kg. In some embodiments of any of the methods described herein, maintaining the osmolality of the first, second, and third liquid culture medium in the vessel includes adjusting the flow rate of the third liquid culture medium during the period of time. In some embodiments of any of the methods described herein, the adjusting step includes increasing the flow rate of the third liquid culture medium at some point during the period of time. In some embodiments of any of the methods described herein, the adjusting step includes decreasing the flow rate of the third liquid culture medium at some point during the period of time. In some embodiments of any of the methods described herein, the method further includes adjusting one or both of the pH and the pCO₂ of the first, second, and third liquid culture medium in the vessel at some point during the period of time.

In some embodiments, the method includes increasing the pCO₂ and increasing the pH of the first, second, and third liquid culture medium in the vessel over the period of time. In some embodiments, the pCO₂ and the pH of the first, second, and third liquid culture medium in the vessel are increased based on the viable cell density over the period of time. In some embodiments of any of the methods described herein, a viable cell density of greater than 60×10⁶ cells/mL in the first, second, and third liquid culture medium is achieved during the period of time. In some embodiments, a viable cell density of greater than 100×10⁶ cells/mL in the first, second, and third liquid culture medium is achieved during the period of time. In some embodiments, a viable cell density of greater than 120×10⁶ cells/mL in the first, second, and third liquid culture medium is achieved during the period of time.

In some embodiments of any of the methods described herein, the method further includes monitoring the osmolality of the first, second, and third liquid culture medium in the vessel during the period of time. In some embodiments of any of the methods described herein, the removing of the first volume of the first liquid culture medium, the adding of the second volume of the second liquid culture medium, and the adding of the third volume during the period of time is performed simultaneously. In some embodiments of any of the methods described herein, the removing of the first volume of the first liquid culture medium, the adding of the second volume of the second liquid culture medium, and the adding of the third volume during the period of time is performed continuously. In some embodiments of any of the methods described herein, the removing of the first volume of the first liquid culture medium, the adding of the second volume of the second liquid culture medium, and the adding of the third volume over the period of time is performed periodically. In some embodiments of any of the methods described herein, the recombinant protein is secreted into the first, second, and/or third liquid culture medium. In some embodiments, the recombinant protein is recovered from the first, second, and/or third liquid culture medium.

In some embodiments of any of the methods described herein, the mammalian cell is a CHO cell. In some embodiments of any of the methods described herein, the vessel is a bioreactor. In some embodiments, the bioreactor is a perfusion bioreactor. In some embodiments of any of the methods described herein, the recombinant protein is an immunoglobulin, an enzyme, a growth factor, a protein fragment, or an engineered protein. In some embodiments of any of the methods described herein, the recombinant protein is recovered from the mammalian cell.

In some embodiments of any of the methods described herein, the method further includes isolating the recombinant protein. In some embodiments, the method further includes formulating the isolated recombinant protein.

Provided herein are recombinant proteins produced by any of the methods described herein. Also provided herein are pharmaceutical compositions including any of the recombinant proteins described herein, and kits including any of the pharmaceutical compositions described herein.

Also provided herein are methods of treating a subject in need thereof including administering a therapeutically effective amount of any of the pharmaceutical compositions described herein to the subject.

As used herein, the word “a” before a noun represents one or more of the particular noun. For example, the phrase “a mammalian cell” represents “one or more mammalian cells.”

The term “mammalian cell” means any cell from or derived from any mammal (e.g., a human, a hamster, a mouse, a green monkey, a rat, a pig, a cow, or a rabbit). For example, a mammalian cell can be an immortalized cell. In some embodiments, the mammalian cell is a differentiated or undifferentiated cell. Non-limiting examples of mammalian cells are described herein. Additional examples of mammalian cells are known in the art.

The term “culturing” or “cell culturing” means the maintenance or proliferation of a mammalian cell under a controlled set of physical conditions.

The term “culture of mammalian cells,” “culture,” or “cell culture” means a liquid medium containing a plurality of mammalian cells that is maintained or proliferated under a controlled set of physical conditions.

The term “liquid culture medium” or “liquid medium” means a fluid that contains sufficient nutrients to allow a cell (e.g., a mammalian cell) to grow or proliferate in vitro. For example, a liquid medium can contain one or more of: amino acids (e.g., 20 amino acids), a purine (hypoxanthine), a pyrimidine (e.g., thymidine), choline, inositol, thiamine, folic acid, biotin, calcium, niacinamide, pyridoxine, riboflavin, thymidine, cyanocobalamin, pyruvate, lipoic acid, magnesium, glucose, sodium, potassium, iron, copper, zinc, and sodium bicarbonate.

In some embodiments, a liquid culture medium can contain serum from a mammal. In some embodiments, a liquid culture medium does not contain serum or another extract from a mammal (a defined liquid culture medium). In some embodiments, a liquid culture medium can contain trace metals, a mammalian growth hormone, and/or a mammalian growth factor. Another example of liquid culture medium is minimal medium (e.g., a medium containing only inorganic salts, a carbon source, and water). Non-limiting examples of liquid culture medium are described herein. Additional examples of liquid culture medium are known in the art and are commercially available. A liquid culture medium can contain any density of mammalian cells. For example, as used herein, a volume of liquid culture medium removed from a bioreactor can be substantially free of mammalian cells.

The term “animal-derived component free liquid culture medium” means a liquid culture medium that does not contain any components (e.g., proteins or serum) derived from a mammal.

The term “serum-free liquid culture medium” means a liquid culture medium that does not contain a mammalian serum.

The term “serum-containing liquid culture medium” means a liquid culture medium that contains a mammalian serum.

The term “chemically-defined liquid culture medium” is a term of art and means a liquid culture medium in which all of the chemical components are known. For example, a chemically-defined liquid culture medium does not contain fetal bovine serum, bovine serum albumin, or human serum albumin, as these preparations typically contain a complex mix of albumins and lipids.

The term “protein-free liquid culture medium” means a liquid culture medium that does not contain any protein (e.g., any detectable protein).

The term “clarified liquid culture medium” means a liquid culture medium obtained from a bacterial or yeast cell culture that is substantially free (e.g., at least 80%, 85%, 90%, 92%, 94%, 96%, 98%, or 99% free) of bacteria or yeast cells.

The term “agitation” means stirring or otherwise moving a portion of liquid culture medium in a bioreactor. This is performed in order to, e.g., increase the dissolved O₂ concentration in the liquid culture medium in a bioreactor. Agitation can be performed using any art known method, e.g., an instrument or propellor. Exemplary devices and methods that can be used to perform agitation of a portion of the liquid culture medium in a bioreactor are known in the art.

The term “immunoglobulin” means a polypeptide containing an amino acid sequence of at least 15 amino acids (e.g., at least 20, 30, 40, 50, 60, 70, 80, 90, or 100 amino acids) of an immunoglobulin protein (e.g., a variable domain sequence, a framework sequence, or a constant domain sequence). The immunoglobulin may, for example, include at least 15 amino acids of a light chain immunoglobulin, e.g., at least 15 amino acids of a heavy chain immunoglobulin. The immunoglobulin may be an isolated antibody (e.g., an IgG, IgE, IgD, IgA, or IgM). The immunoglobulin may be an antibody fragment, e.g., a Fab fragment, a F(ab′)₂ fragment, or a scFv fragment. The immunoglobulin may also be a bi-specific antibody or a tri-specific antibody, or a dimer, a trimer, a multimer antibody, or a diabody, an Affibody®, or a Nanobody®. The immunoglobulin can also be an engineered protein containing at least one immunoglobulin domain (e.g., a fusion protein). Non-limiting examples of immunoglobulins are described herein and additional examples of immunoglobulins are known in the art. A recombinant immunoglobulin can be produced using any of the methods described herein.

The term “engineered protein” means a polypeptide that is not naturally encoded by an endogenous nucleic acid present within an organism (e.g., a mammal). Examples of engineered proteins include enzymes (e.g., with one or more amino acid substitutions, deletions, insertions, or additions that result in an increase in stability and/or catalytic activity of the engineered enzyme), fusion proteins, antibodies (e.g., divalent antibodies, trivalent antibodies, or a diabody), and antigen-binding proteins that contain at least one recombinant scaffolding sequence.

The term “secreted protein” or “secreted recombinant protein” means a protein (e.g., a recombinant protein) that originally contained at least one secretion signal sequence when it is translated within a mammalian cell, and through, at least in part, enzymatic cleavage of the secretion signal sequence in the mammalian cell, is secreted at least partially into the extracellular space (e.g., a liquid culture medium). Skilled practitioners will appreciate that a “secreted” protein need not dissociate entirely from the cell to be considered a secreted protein.

The term “perfusion bioreactor” means a bioreactor containing a plurality of cells (e.g., mammalian cells) in a first liquid culture medium, wherein the culturing of the cells present in the bioreactor includes periodic or continuous removal of the first liquid culture medium and at the same time or shortly thereafter adding substantially the same volume of a second liquid culture medium to the bioreactor. In some examples, there is an incremental change (e.g., increase or decrease) in the volume of the first liquid culture medium removed and added over incremental periods (e.g., an about 24-hour period, a period of between about 1 minute and about 24-hours, or a period of greater than 24 hours) during the culturing period (e.g., the liquid culture medium refeed rate on a daily basis). The fraction of media removed and replaced each day can vary depending on the particular cells being cultured, the initial seeding density, and the cell density at a particular time. “RV” or “reactor volume” means the volume of the liquid culture medium present at the beginning of the culturing process (e.g., the total volume of the liquid culture medium present after seeding).

“Specific productivity” or “SP” is a term of art and as used herein refers to the mass or enzymatic activity of a recombinant therapeutic protein produced per mammalian cell per day. The SP for a recombinant therapeutic antibody is usually measured as mass/cell/day. The SP for a recombinant therapeutic enzyme is usually measured as units/cell/day or (units/mass)/cell/day.

“Volume productivity” or “VP” is a term of art and as used herein refers to the mass or enzymatic activity of recombinant therapeutic protein produced per volume of culture (e.g., per L of bioreactor, vessel, or tube volume) per day. The VP for a recombinant therapeutic antibody is usually measured as mass/L/day. The VP for a recombinant therapeutic enzyme is usually measured as units/L/day or mass/L/day.

The phrases “poloxamer at a concentration” and “poloxamer concentration” are used interchangeably herein. The phrases “poloxamer of X g/L or more” and “poloxamer of X g/L or at a greater concentration X g/L” are used interchangeably herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a graph of the viable cell density (VCD) (million cells/mL) over time in a batch cell culture run performed at different osmolalities (mOsm/kg) in shake flasks. (n=2). The average osmolality from days 0, 4, and 7 is shown in mOsm.

FIG. 1B is a graph of the viable cell density (VCD) (million cells/mL) over time in a batch cell culture run performed at different osmolalities (mOsm/kg) in Ambr15 microbioreactors. (n=2). The average osmolality from days 0, 2, 5, 7, and 9 is shown in mOsm.

FIG. 2A is a graph of the average cell diameter (μm) at day 4 over osmolalities (mOsm/kg) in shake flasks.

FIG. 2B is a graph of the average cell diameter (μm) at day 5 over osmolalities (mOsm/kg) in Ambr15 microbioreactors.

FIG. 3A is a graph of the osmolality (mOsm/kg) over time in a perfusion cell culture run performed in bioreactors. Run V22 (“V22”) had a ratio of concentrated cell culture medium feed to water feed that resulted in high osmolality in the bioreactor.

FIG. 3B is a graph of the viable cell density (VCD) (million cells/mL) (solid line) and the Vi-CELL average diameter (μm) (dashed line) over time in a perfusion cell culture run performed in a bioreactor. Run V22 (“V22”) experienced high osmolality which led to a decrease in growth rate and a decrease in viability.

FIG. 3C is a graph of the harvest volume productivity (VP) (g/L/day) (solid line) and the harvest titer (g/L) (dashed line) over time in a perfusion cell culture run performed in a bioreactor. Run V22 (“V22”) had high osmolality, which decreased productivity.

FIG. 3D is a graph of the viability (%) (solid line) and the lactate dehydrogenase (LDH) production (U/L/day) (dashed line) over time in a perfusion cell culture run performed in a bioreactor. Run V22 (“V22”) had high osmolality, leading to high LDH production which indicates poor culture health.

FIG. 3E is a graph of the glucose concentration (g/L) (solid line) and the lactate concentration (g/L) (dashed line) over time in a perfusion cell culture run performed in a bioreactor. Run V22 (“V22”) had high osmolality resulting in increased lactate production.

FIG. 4A is a graph of the viable cell density (VCD) (million cells/mL) (solid line) and the viability (%) (dashed line) over time in a perfusion cell culture run performed in a Protein 3 bioreactor. High osmolality led to decreased VCD and culture viability.

FIG. 4B is a graph of the lactate concentration (g/L) (solid line) and the lactate osmolality (mOsm/kg) (dashed line) over time in a perfusion cell culture run performed in a bioreactor. High osmolality resulted in increased lactate production.

FIG. 5A is a graph of the osmolality (mOsm/kg) over time in a set of perfusion cell culture bioreactors showing different osmolality profiles.

FIG. 5B is a graph of the viable cell density (VCD) (million cells/mL) (solid line) and the Vi-CELL average diameter (μm) (dashed line) over time in a perfusion cell culture run performed in bioreactors. Differences in VCD profiles correspond to different culture osmolalities.

FIG. 6A is a graph of the osmolality (mOsm/kg) over conductivity (mS/cm) in a perfusion cell culture run performed in a bioreactor using all data.

FIG. 6B is a graph of the osmolality (mOsm/kg) over conductivity (mS/cm) in a perfusion cell culture run performed in a bioreactor using data characterized by reactor scale and the phase of the culture (low or high biomass).

FIG. 7 is a graph of the conductivity (mS/cm) (solid line) and osmolality (mOsm/kg) (individual data points) over time in a perfusion cell culture run performed in a bioreactor with automated conductivity feedback control.

FIG. 8A is a graph of the osmolality (mOsm/kg) over time in a perfusion cell culture run performed in 10 L bioreactors with automated conductivity feedback control.

FIG. 8B is a graph of the viable cell density (VCD) (million cells/mL) (solid line) and the Vi-CELL average diameter (μm) (dashed line) over time in a perfusion cell culture run performed in 10 L bioreactors showing consistent VCD profiles.

FIG. 9 is a graph of the conductivity (mS/cm) over osmolality (mOsm/kg) of osmolality standards measured with different probe types.

FIG. 10 is a graph of the osmolality (mOsm/kg) over time in a perfusion cell culture run performed in a 100 L bioreactor with predicted osmolality from Raman spectroscopy measurements.

FIG. 11 is a graph of the viable cell density (VCD) (million cells/mL) over osmolality (mOsm/kg) in a 10 L bioreactor on day 30 of a 60-day perfusion cell culture process.

### DETAILED DESCRIPTION

Provided herein are methods of perfusion culturing a mammalian cell that include: providing a vessel containing a mammalian cell disposed in a first liquid culture medium having an osmolality of about 270 mOsm/kg to about 380 mOsm/kg; incubating the mammalian cell for a period of time at about 32° C. to about 39° C.; and during the period of time, continuously or periodically removing a first volume of the first liquid culture medium and adding to the first liquid culture medium a second volume of a second liquid culture medium, wherein the first and second volumes are about equal and the osmolality of the first and second liquid culture medium in the vessel is maintained at about 270 mOsm/kg to about 380 mOsm/kg over the period of time. Some embodiments of these methods further include continuously or periodically adding a third volume of an aqueous solution to the first and the second liquid culture medium in the vessel during the period of time, wherein the first volume and the sum of the second and third volumes are about equal and the osmolality of the first, second, and third liquid culture medium in the vessel is maintained at about 270 mOsm/kg to about 380 mOsm/kg over the period of time. Some embodiments of these methods further include continuously or periodically adding to the first liquid culture medium a third volume of a third liquid culture medium, wherein the first volume and the sum of the second and third volumes are about equal and the osmolality of the first, second, and third liquid culture medium in the vessel is maintained at about 270 mOsm/kg to about 380 mOsm/kg over the period of time. In some examples, the third liquid culture medium includes sodium chloride (NaCl) or potassium chloride (KCl). In some examples, the maintenance of the osmolality of the first, second, and third liquid culture medium in the vessel includes adjusting the flow rate of the third liquid culture medium during the period of time. In other examples, the osmolality is controlled by the flow rate of the third liquid culture medium. In other examples, the third liquid culture medium can have an osmolality of about 0 mOsm/kg to about 270 mOsm/kg. For example, when the second liquid culture medium has an osmolality of greater than 380 mOsm/kg, a third liquid culture medium having an osmolality of about 0 mOsm/kg to about 100 mOsm/kg can be used. In some examples, the maintenance of the osmolality in the first, second, and third liquid culture medium in the vessel includes adjusting the flow rate of the third liquid culture medium during the period of time.

Also provided herein are methods of producing a recombinant protein that include: providing a vessel containing a mammalian cell containing a nucleic acid encoding a recombinant protein disposed in a first liquid culture medium having an osmolality of about 270 mOsm/kg to about 380 mOsm/kg; incubating the mammalian cell for a period of time at about 32° C. to about 39° C.; and during the period of time, continuously or periodically removing a first volume of the first liquid culture medium and adding to the first liquid culture medium a second volume of a second liquid culture medium, wherein the first and second volumes are about equal and the osmolality of the first and second liquid culture medium in the vessel is maintained at about 270 mOsm/kg to about 380 mOsm/kg over the period of time; and recovering the recombinant protein from the mammalian cell or from the first and/or second liquid culture medium. Some embodiments of these methods further include continuously or periodically adding a third volume of an aqueous solution to the first and the second liquid culture medium in the vessel during the period of time, wherein the first volume and the sum of the second and third volumes are about equal and the osmolality of the first, second, and third liquid culture medium in the vessel is maintained at about 270 mOsm/kg to about 380 mOsm/kg over the period of time. Some embodiments of these methods further include continuously or periodically adding to the first liquid culture medium a third volume of a third liquid culture medium, wherein the first volume and the sum of the second and third volumes are about equal and the osmolality of the first, second, and third liquid culture medium in the vessel is maintained at about 270 mOsm/kg to about 380 mOsm/kg over the period of time. In some examples, the third liquid culture medium includes sodium chloride (NaCl) or potassium chloride (KCl). In some examples, the maintenance of the osmolality of the first, second, and third liquid culture medium in the vessel includes adjusting the flow rate of the third liquid culture medium during the period of time. In other examples, the third liquid culture medium can have an osmolality of about 0 mOsm/kg to about 270 mOsm/kg. For example, when the second liquid culture medium has an osmolality of greater than 380 mOsm/kg, a third liquid culture medium having an osmolality of about 0 mOsm/kg to about 100 mOsm/kg can be used. In some examples, the maintenance of the osmolality in the first, second, and third liquid culture medium in the vessel includes adjusting the flow rate of the third liquid culture medium during the period of time.

In some embodiments, the methods provided herein can achieve a cell culture having a viable cell density of greater than 60×10⁶ cells/mL in the first and second liquid culture medium, or in the first, second, and third liquid culture medium during the period of time. Methods for determining the viable cell density of a cell culture are well known in the art.

In some embodiments, the methods described herein provide for a cell culture having a percentage cell viability that is greater than 75% (e.g., greater than 80%, greater than 85%, or greater than 90%) over a culturing period of at least 5 days (e.g., at least 10 days, at least 15 days, at least 20 days, at least 25 days, at least 30 days, at least 35 days, at least 40 days, at least 45 days, at least 50 days, at least 55 days, at least 60 days, at least 65 days, or at least 70 days).

### Culture Volumes (10 L-10,000 L Fed-Batch, 50-500 L-2000 L, 50 L, 500 L, 1000 L, 2000 L)

Any of the methods described herein can include incubating providing a vessel (e.g., any of the vessels described herein) containing a mammalian cell (e.g., any of the mammalian cells described herein) in a first liquid culture medium having a volume (at the start of the period of time) of between about 10 L and about 10,000 L (e.g., between about 10 L and about 8,000 L, between about 10 L and about 7,000 L, between about 10 L and about 6,000 L, between about 10 L and about 5,000 L, between about 10 L and about 4,000 L, between about 10 L and about 3,000 L, between about 10 L and about 2,500 L, between about 10 L and about 2,000 L, between about 10 L and about 1,500 L, between about 10 L and about 1,000 L, between about 10 L and about 500 L, between about 10 L and about 200 L, between about 10 L and about 100 L, between about 20 L and about 10,000 L, between about 20 L and about 8,000 L, between about 20 L and about 7,000 L, between about 20 L and about 6,000 L, between about 20 L an about 5,000 L, between about 20 L and about 4,000 L, between about 20 L and about 3,000 L, between about 20 L and about 2,500 L, between about 20 L and about 2,000 L, between about 20 L and about 1,500 L, between about 20 L and about 1,000 L, between about 20 L and about 500 L, between about 20 L and about 200 L, between about 20 L and about 100 L, between about 100 L and about 10,000 L, between about 100 L and about 8,000 L, between about 100 L and about 7,000 L, between about 100 L and about 6,000 L, between about 100 L and about 5,000 L, between about 100 L and about 4,000 L, between about 100 L and about 3,000 L, between about 100 L and about 2,000 L, between about 100 L and about 1,500 L, between about 100 L and about 1,000 L, between about 100 L and about 800 L, between about 100 L and about 700 L, between about 100 L and about 600 L, between about 100 L and about 500 L, between about 100 L and about 400 L, between about 100 L and about 300 L, between about 100 L and about 200 L, between about 500 L and about 10,000 L, between about 500 L and about 8,000 L, between about 500 L and about 7,000 L, between about 500 L and about 6,000 L, between about 500 L and about 5,000 L, between about 500 L and about 4,000 L, between about 500 L and about 3,000 L, between about 500 L and about 2,000 L, between about 500 L and about 1,500 L, between about 500 L and about 1,000 L, between about 500 L and about 750 L, between about 1,000 L and about 10,000 L, between about 1,000 L and about 8,000 L, between about 1,000 L and about 7,000 L, between about 1,000 L and about 6,000 L, between about 1,000 L and about 5,000 L, between about 1,000 L and about 4,000 L, between about 1,000 L and about 3,000 L, between about 1,000 L and about 2,000 L, between about 1,000 L and about 1,500 L, between about 2,000 L and about 10,000 L, between about 2,000 L and about 8,000 L, between about 2,000 L and about 7,000 L, between about 2,000 L and about 6,000 L, between about 2,000 L and about 5,000 L, between about 2,000 L and about 4,000 L, between about 2,000 L and about 3,000 L, between about 3,000 L and about 10,000 L, between about 3,000 L and about 8,000 L, between about 3,000 L and about 7,000 L, between about 3,000 L and about 6,000 L, between about 3,000 L and about 5,000 L, between about 3,000 L and about 4,000 L, between about 4,000 L and about 10,000 L, between about 4,000 L and about 8,000 L, between about 4,000 L and about 7,000 L, between about 4,000 L and about 6,000 L, between about 4,000 L and about 5,000 L, between about 5,000 L and about 10,000 L, between about 5,000 L and about 8,000 L, between about 5,000 L and about 7,000 L, between about 5,000 L and about 6,000 L, between about 6,000 L and about 10,000 L, between about 6,000 L and about 8,000 L, between about 6,000 L and about 7,000 L, between about 7,000 L and about 10,000 L, between about 7,000 L and about 8,000 L, or between about 8,000 L and about 10,000 L, or 10 L, 20 L, 50 L, 100 L, 500 L, 1,000 L, or 2,000 L). The step of incubating the mammalian cell is performed under conditions that allow for cell maintenance and proliferation.

The first liquid culture medium can have an osmolality of about 270 mOsm/kg to about 320 mOsm/kg (e.g., about 270 mOsm/kg to about 315 mOsm/kg, about 270 mOsm/kg to about 310 mOsm/kg, about 270 mOsm/kg to about 305 mOsm/kg, about 270 mOsm/kg to about 300 mOsm/kg, about 270 mOsm/kg to about 295 mOsm/kg, about 270 mOsm/kg to about 290 mOsm/kg, about 270 mOsm/kg to about 285 mOsm/kg, about 270 mOsm/kg to about 280 mOsm/kg, about 270 mOsm/kg to about 275 mOsm/kg, about 275 mOsm/kg to about 320 mOsm/kg, about 275 mOsm/kg to about 315 mOsm/kg, about 275 mOsm/kg to about 310 mOsm/kg, about 275 mOsm/kg to about 305 mOsm/kg, about 275 mOsm/kg to about 300 mOsm/kg, about 275 mOsm/kg to about 295 mOsm/kg, about 275 mOsm/kg to about 290 mOsm/kg, about 275 mOsm/kg to about 285 mOsm/kg, about 275 mOsm/kg to about 280 mOsm/kg, about 280 mOsm/kg to about 320 mOsm/kg, about 280 mOsm/kg to about 315 mOsm/kg, about 280 mOsm/kg to about 310 mOsm/kg, about 280 mOsm/kg to about 305 mOsm/kg, about 280 mOsm/kg to about 300 mOsm/kg, about 280 mOsm/kg to about 295 mOsm/kg, about 280 mOsm/kg to about 290 mOsm/kg, about 280 mOsm/kg to about 285 mOsm/kg, about 285 mOsm/kg to about 320 mOsm/kg, about 285 mOsm/kg to about 315 mOsm/kg, about 285 mOsm/kg to about 310 mOsm/kg, about 285 mOsm/kg to about 305 mOsm/kg, about 285 mOsm/kg to about 300 mOsm/kg, about 285 mOsm/kg to about 295 mOsm/kg, about 285 mOsm/kg to about 290 mOsm/kg, about 290 mOsm/kg to about 320 mOsm/kg, about 290 mOsm/kg to about 315 mOsm/kg, about 290 mOsm/kg to about 310 mOsm/kg, about 290 mOsm/kg to about 305 mOsm/kg, about 290 mOsm/kg to about 300 mOsm/kg, about 290 mOsm/kg to about 295 mOsm/kg, about 295 mOsm/kg to about 320 mOsm/kg, about 295 mOsm/kg to about 315 mOsm/kg, about 295 mOsm/kg to about 310 mOsm/kg, about 295 mOsm/kg to about 305 mOsm/kg, about 295 mOsm/kg to about 300 mOsm/kg, about 300 mOsm/kg to about 320 mOsm/kg, about 300 mOsm/kg to about 315 mOsm/kg, about 300 mOsm/kg to about 310 mOsm/kg, about 300 mOsm/kg to about 305 mOsm/kg, about 305 mOsm/kg to about 320 mOsm/kg, about 305 mOsm/kg to about 315 mOsm/kg, about 305 mOsm/kg to about 310 mOsm/kg, about 310 mOsm/kg to about 320 mOsm/kg, about 310 mOsm/kg to about 315 mOsm/kg, or about 315 mOsm/kg to about 320 mOsm/kg). Non-limiting aspects and examples of first liquid culture medium are described herein.

In some embodiments of any of the methods described herein, the second volume of the second liquid culture medium (e.g., any of the exemplary second liquid culture medium described herein) added to the first liquid culture medium (e.g., any of the exemplary first liquid culture medium described herein) (and optionally the third liquid culture medium) is increased during the period of time by at least 10% (e.g., at least 15%, at least 20%, at least 25%, at least 30%, at least 45%, at least 50%, at least 75%, at least 100%, at least 200 at least 300%, at least 400%, or at least 500%, or about 10% to about 500%, about 10% to about 400%, about 10% to about 300%, about 10% to about 200%, about 10% to about 100%, about 10% to about 50%, about 10% to about 20%, about 20% to about 500 about 20% to about 400%, about 20% to about 300%, about 20% to about 200%, about 20% to about 100%, about 20% to about 50%, about 20% to about 40%, about 20% to about 30%, about 30% to about 500%, about 30% to about 400%, about 30% to about 300%, about 30% to about 200%, about 30% to about 100%, about 30% to about 50%, about 30% to about 40%, about 40% to about 500about 40% to about 400%, about 40% to about 300%, about 40% to about 200%, about 40% to about 100%, about 40% to about 50%, about 50% to about 500%, about 50% to about 400%, about 50% to about 300%, about 50% to about 200%, about 50% to about 100%, about 50% to about 60%, about 60% to about 500%, about 60% to about 400%, about 60% to about 300%, about 60% to about 200%, about 60% to about 100%, about 60% to about 80%, about 60% to about 70%, about 70% to about 500%, about 70% to about 400%, about 70% to about 300%, about 70% to about 200%, about 70% to about 100%, about 70% to about 80%, about 80% to about 500%, about 80% to about 400%, about 80% to about 300%, about 80% to about 200%, about 80% to about 100%, about 80% to about 90%, about 90% to about 500%, about 90% to about 400%, about 90% to about 300%, about 90% to about 200%, about 90% to about 100%, about 100% to about 500%, about 100% to about 400%, about 100% to about 300%, about 100% to about 200%, about 100% to about 150%, about 200% to about 500%, about 200% to about 400%, about 200% to about 300%, about 200% to about 250%, about 300% to about 500%, about 300% to about 400%, about 400% to about 500%, or about 450% to about 500%) as compared to the second volume of the second liquid culture medium added at an earlier time point during the period of time.

In some embodiments of any of the methods described herein, the second volume of the second liquid culture medium (e.g., any of the exemplary second liquid culture medium described herein) added to the first liquid culture medium (e.g., any of the first liquid culture medium described herein) (and optionally to the third liquid culture medium) is increased by at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 120%, at least 140%, at least 160%, at least 180%, at least 200%, at least 220%, at least 240%, at least 260%, at least 280%, at least 300%, at least 320%, at least 340%, at least 360%, at least 380%, at least 400%, at least 420%, at least 440%, at least 460%, at least 480%, or at least 500%, or about 10% to about 500% (e.g., or any of the subranges of this range described herein) as compared to the second volume of the second liquid culture medium added at an earlier time point during the period of time, based on the viable cell density over the period of time.

In some embodiments of any of the methods described herein, the flow rate of the second liquid culture medium added to the first liquid culture medium (and optionally the third liquid culture medium) during the period of time is increased by at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 120%, at least 140%, at least 160%, at least 180%, at least 200%, at least 220%, at least 240%, at least 260%, at least 280%, or at least 300%, or about 10% to about 300%, about 10% to about 250%, about 10% to about 200%, about 10% to about 150%, about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 300%, about 20% to about 250%, about 20% to about 200%, about 20% to about 150%, about 20% to about 100%, about 20% to about 90%, about 20% to about 80%, about 20% to about 70%, about 20% to about 60%, about 20% to about 50%, about 20% to about 40%, about 20% to about 30%, about 30% to about 300%, about 30% to about 250%, about 30% to about 200%, about 30% to about 150%, about 30% to about 100%, about 30% to about 90%, about 30% to about 80%, about 30% to about 70%, about 30% to about 60%, about 30% to about 50%, about 30% to about 40%, about 40% to about 300%, about 40% to about 250%, about 40% to about 200%, about 40% to about 150%, about 40% to about 100%, about 40% to about 90%, about 40% to about 80%, about 40% to about 70%, about 40% to about 60%, about 40% to about 50%, about 50% to about 300%, about 50% to about 250%, about 50% to about 200%, about 50% to about 150%, about 50% to about 100%, about 50% to about 90%, about 50% to about 80%, about 50% to about 70%, about 50% to about 60%, about 60% to about 300%, about 60% to about 250%, about 60% to about 200%, about 60% to about 150%, about 60% to about 100%, about 60% to about 90%, about 60% to about 80%, about 60% to about 70%, about 70% to about 300%, about 70% to about 250%, about 70% to about 200%, about 70% to about 150%, about 70% to about 100%, about 70% to about 90%, about 70% to about 80%, about 80% to about 300%, about 80% to about 250%, about 80% to about 200%, about 80% to about 150%, about 80% to about 100%, about 80% to about 90%, about 90% to about 300%, about 90% to about 250%, about 90% to about 200%, about 90% to about 150%, about 90% to about 100%, about 100% to about 300%, about 100% to about 250%, about 100% to about 200%, about 100% to about 150%, about 150% to about 300%, about 150% to about 250%, about 150% to about 200%, about 200% to about 300%, about 200% to about 250%, or about 250% to about 300%) as compared to the flow rate of the second liquid culture medium at an earlier time point during the period of time.

In some embodiments of any of the methods described herein, the flow rate of the second liquid culture medium added to the first liquid culture medium (and optionally the third liquid culture medium) during the period of time is decreased by at least 10% (e.g., at least 12%, at least 14%, at least 15%, at least 16%, at least 18%, at least 20%, at least 22%, at least 24%, at least 25%, at least 26%, at least 28%, at least 30%, at least 32%, at least 34%, at least 35%, at least 36%, at least 38%, at least 40%, at least 42%, at least 44%, at least 45%, at least 46%, at least 48%, at least 50%, at least 52%, at least 54%, at least 55%, at least 56%, at least 58%, at least 60%, at least 62%, at least 64%, at least 65%, at least 66%, at least 68%, at least 70%, at least 72%, at least 74%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, or at least 99%, or about 1% to about 100%, about 1% to about 90%, about 1% to about 80%, about 1% to about 70%, about 1% to about 60%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 100%, about 5% to about 90%, about 5% to about 80%, about 5% to about 70%, about 5% to about 60%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 100%, about 10% to about 90%, about 10% to about 80%, about 10% to about 70%, about 10% to about 60%, about 10% to about 50%, about 10% to about 45%, about 10% to about 40%, about 10% to about 35%, about 10% to about 30%, about 10% to about 25%, about 10% to about 20%, about 10% to about 15%, about 15% to about 100%, about 15% to about 90%, about 15% to about 80%, about 15% to about 70%, about 15% to about 60%, about 15% to about 50%, about 15% to about 45%, about 15% to about 40%, about 15% to about 35%, about 15% to about 30%, about 15% to about 25%, about 15% to about 20%, about 20% to about 100%, about 20% to about 90%, about 20% to about 80%, about 20% to about 70%, about 20% to about 60%, about 20% to about 50%, about 20% to about 45%, about 20% to about 40%, about 20% to about 35%, about 20% to about 30%, about 20% to about 25%, about 25% to about 100%, about 25% to about 90%, about 25% to about 80%, about 25% to about 70%, about 25% to about 60%, about 25% to about 50%, about 25% to about 45%, about 25% to about 40%, about 25% to about 35%, about 25% to about 30%, about 30% to about 100%, about 30% to about 90%, about 30% to about 80%, about 30% to about 70%, about 30% to about 60%, about 30% to about 50%, about 30% to about 45%, about 30% to about 40%, about 30% to about 35%, about 35% to about 100%, about 35% to about 90%, about 35% to about 80%, about 35% to about 70%, about 35% to about 60%, about 35% to about 50%, about 35% to about 45%, about 35% to about 40%, about 40% to about 100%, about 40% to about 90%, about 40% to about 80%, about 40% to about 70%, about 40% to about 60%, about 40% to about 50%, about 40% to about 45%, about 45% to about 100%, about 45% to about 90%, about 45% to about 80%, about 45% to about 70%, about 45% to about 60%, about 45% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%) as compared to the flow rate of the second liquid culture medium at an earlier time point during the period of time. Non-limiting aspects and examples of second liquid culture medium are described herein.

### Period of Time (30-60 Days; 7-90 Days, 45 Days, 60 Days)

In some examples of any of the methods described herein, the period of time is between about 5 days to about 100 days (e.g., between about 5 days and about 95 days, between about 5 days and about 90 days, between about 5 days and about 85 days, between about 5 days and about 80 days, between about 5 days and about 75 days, between about 5 days and about 70 days, between about 5 days and about 65 days, between about 5 days and about 60 days, between about 5 days and about 55 days, between about 5 days and about 50 days, between about 5 days and about 45 days, between about 5 days and about 40 days, between about 5 days and about 35 days, between about 5 days and about 30 days, between about 5 days and about 25 days, between about 5 days and about 20 days, between about 5 days and about 15 days, between about 5 days and about 10 days, between about 5 days and about 7 days, between about 7 days and about 95 days, between about 7 days and about 90 days, between about 7 days and about 85 days, between about 7 days and about 80 days, between about 7 days and about 75 days, between about 7 days and about 70 days, between about 7 days and about 65 days, between about 7 days and about 60 days, between about 7 days and about 55 days, between about 7 days and about 50 days, between about 7 days and about 45 days, between about 7 days and about 40 days, between about 7 days and about 35 days, between about 7 days and about 30 days, between about 7 days and about 25 days, between about 7 days and about 20 days, between about 7 days and about 15 days, between about 7 days and about 10 days, between about 10 days and about 100 days, between about 10 days and about 95 days, between about 10 days and about 90 days, between about 10 days and about 85 days, between about 10 days and about 80 days, between about 10 days and about 75 days, between about 10 days and about 70 days, between about 10 days and about 65 days, between about 10 days and about 60 days, between about 10 days and about 55 days, between about 10 days and about 50 days, between about 10 days and about 45 days, between about 10 days and about 40 days, between about 10 days and about 35 days, between about 10 days and about 30 days, between about 10 days and about 25 days, between about 10 days and about 20 days, between about 10 days and about 15 days, between about 15 days and about 100 days, between about 15 days and about 95 days, between about 15 days and about 90 days, between about 15 days and about 85 days, between about 15 days and about 80 days, between about 15 days and about 75 days, between about 15 days and about 70 days, between about 15 days and about 65 days, between about 15 days and about 60 days, between about 15 days and about 55 days, between about 15 days and about 50 days, between about 15 days and about 45 days, between about 15 days and about 40 days, between about 15 days and about 35 days, between about 15 days and about 30 days, between about 15 days and about 25 days, between about 15 days and about 20 days, between about 30 days and about 100 days, between about 30 days and about 90 days, between about 30 days and about 80 days, between about 30 days and about 70 days, between about 30 days and about 60 days, between about 30 days and about 50 days, between about 30 days and about 40 days, between about 40 days and about 100 days, between about 40 days and about 90 days, between about 40 days and about 80 days, between about 40 days and about 70 days, between about 40 days and about 60 days, between about 40 days and about 50 days, between about 50 days and about 100 days, between about 50 days and about 90 days, between about 50 days and about 80 days, between about 50 days and about 70 days, between about 50 days and about 60 days, between about 60 days and about 100 days, between about 60 days and about 90 days, between about 60 days and about 80 days, between about 60 days and about 70 days, between about 70 days and about 100 days, between about 70 days and about 90 days, between about 70 days and about 80 days, between about 80 days and about 100 days, between about 80 days and about 90 days, or between about 90 days and about 100 days, or 7 days, 21 days, 28 days, 30 days, 31 days, 45 days, 60 days, or 90 days).

### Mammalian Cells

The mammalian cell cultured (e.g., perfusion cultured) in the methods provided herein can be a cell that grows in suspension or an adherent cell. Non-limiting examples of mammalian cells that can be cultured in any of the methods described herein include: Chinese hamster ovary (CHO) cells (e.g., CHO DG44 cells or CHO-K1s cells), Sp2.0, myeloma cells (e.g., NS/0), B-cells, hybridoma cells, T-cells, human embryonic kidney (HEK) cells (e.g., HEK 293E and HEK 293F), African green monkey kidney epithelial cells (Vero) cells, an NSO cell, a baby hamster kidney (BHK) cell, a PerC6 cell, a Vero cells, a HT-1080 cell, and Madin-Darby Canine (Cocker Spaniel) kidney epithelial cells (MDCK) cells. In some examples where an adherent cell is cultured, the culture can also contain a plurality of microcarriers (e.g., microcarriers that contain one or more pores). Additional mammalian cells that can be cultured in any of the methods described herein are known in the art.

The mammalian cell can contain a recombinant nucleic acid (e.g., a nucleic acid stably integrated in the mammalian cell's genome) that encodes a recombinant protein (e.g., any of the exemplary recombinant proteins described herein). Non-limiting examples of recombinant nucleic acids that encode exemplary recombinant proteins are described below, as are recombinant proteins that can be produced using the methods described herein. In some instances, the mammalian cell that is cultured in a bioreactor (e.g., any of the bioreactors described herein) was derived from a larger culture.

A nucleic acid encoding a recombinant protein can be introduced into a mammalian cell using a wide variety of methods known in molecular biology and molecular genetics. Non-limiting examples include transfection (e.g., lipofection), transduction (e.g., lentivirus, adenovirus, or retrovirus infection), and electroporation. In some instances, the nucleic acid that encodes a recombinant protein is not stably integrated into a chromosome of the mammalian cell (transient transfection), while in others the nucleic acid is integrated. Alternatively or in addition, the nucleic acid encoding a recombinant protein can be present in a plasmid and/or in a mammalian artificial chromosome (e.g., a human artificial chromosome). Alternatively or in addition, the nucleic acid can be introduced into the cell using a viral vector (e.g., a lentivirus, retrovirus, or adenovirus vector). The nucleic acid can be operably linked to a promoter sequence (e.g., a strong promoter, such as a β-actin promoter and CMV promoter, or an inducible promoter). A vector containing the nucleic acid can, if desired, also contain a selectable marker (e.g., a gene that confers hygromycin, puromycin, or neomycin resistance to the mammalian cell).

In some instances, the recombinant protein is a secreted protein and is released by the mammalian cell into the extracellular medium (e.g., the first and/or second liquid medium in perfusion culturing or the first liquid medium and/or feed liquid medium in feed batch culturing). For example, a nucleic acid sequence encoding a soluble recombinant protein can contain a sequence that encodes a secretion signal peptide at the N- or C-terminus of the recombinant protein, which is cleaved by an enzyme present in the mammalian cell, and subsequently released into the extracellular medium (e.g., the first and/or second liquid medium).

### Vessels (10 L-10,000 L Fed Batch, 50 L-500 L-2,000 L, 50 L, 500 L, 1,000 L, 2,000 L)

The culturing step in any of the methods described herein can be performed using a vessel, e.g., a bioreactor (e.g., a perfusion bioreactor). A bioreactor (e.g., a perfusion bioreactor) can have an internal volume (capacity) of between about 10 L and about 10,000 L (e.g., between about 10 L and about 8,000 L, between about 10 L and about 7,000 L, between about 10 L and about 6,000 L, between about 10 L and about 5,000 L, between about 10 L and about 4,000 L, between about 10 L and about 3,000 L, between about 10 L and about 2,500 L, between about 10 L and about 2,000 L, between about 10 L and about 1,500 L, between about 10 L and about 1,000 L, between about 10 L and about 500 L, between about 10 L and about 200 L, between about 10 L and about 100 L, between about 20 L and about 10,000 L, between about 20 L and about 8,000 L, between about 20 L and about 7,000 L, between about 20 L and about 6,000 L, between about 20 L an about 5,000 L, between about 20 L and about 4,000 L, between about 20 L and about 3,000 L, between about 20 L and about 2,500 L, between about 20 L and about 2,000 L, between about 20 L and about 1,500 L, between about 20 L and about 1,000 L, between about 20 L and about 500 L, between about 20 L and about 200 L, between about 20 L and about 100 L, between about 100 L and about 10,000 L, between about 100 L and about 8,000 L, between about 100 L and about 7,000 L, between about 100 L and about 6,000 L, between about 100 L and about 5,000 L, between about 100 L and about 4,000 L, between about 100 L and about 3,000 L, between about 100 L and about 2,000 L, between about 100 L and about 1,500 L, between about 100 L and about 1,000 L, between about 100 L and about 800 L, between about 100 L and about 700 L, between about 100 L and about 600 L, between about 100 L and about 500 L, between about 100 L and about 400 L, between about 100 L and about 300 L, between about 100 L and about 200 L, between about 500 L and about 10,000 L, between about 500 L and about 8,000 L, between about 500 L and about 7,000 L, between about 500 L and about 6,000 L, between about 500 L and about 5,000 L, between about 500 L and about 4,000 L, between about 500 L and about 3,000 L, between about 500 L and about 2,000 L, between about 500 L and about 1,500 L, between about 500 L and about 1,000 L, between about 500 L and about 750 L, between about 1,000 L and about 10,000 L, between about 1,000 L and about 8,000 L, between about 1,000 L and about 7,000 L, between about 1,000 L and about 6,000 L, between about 1,000 L and about 5,000 L, between about 1,000 L and about 4,000 L, between about 1,000 L and about 3,000 L, between about 1,000 L and about 2,000 L, between about 1,000 L and about 1,500 L, between about 2,000 L and about 10,000 L, between about 2,000 L and about 8,000 L, between about 2,000 L and about 7,000 L, between about 2,000 L and about 6,000 L, between about 2,000 L and about 5,000 L, between about 2,000 L and about 4,000 L, between about 2,000 L and about 3,000 L, between about 3,000 L and about 10,000 L, between about 3,000 L and about 8,000 L, between about 3,000 L and about 7,000 L, between about 3,000 L and about 6,000 L, between about 3,000 L and about 5,000 L, between about 3,000 L and about 4,000 L, between about 4,000 L and about 10,000 L, between about 4,000 L and about 8,000 L, between about 4,000 L and about 7,000 L, between about 4,000 L and about 6,000 L, between about 4,000 L and about 5,000 L, between about 5,000 L and about 10,000 L, between about 5,000 L and about 8,000 L, between about 5,000 L and about 7,000 L, between about 5,000 L and about 6,000 L, between about 6,000 L and about 10,000 L, between about 6,000 L and about 8,000 L, between about 6,000 L and about 7,000 L, between about 7,000 L and about 10,000 L, between about 7,000 L and about 8,000 L, or between about 8,000 L and about 10,000 L, or about 10 L, about 20 L, about 50 L, about 100 L, about 500 L, about 1,000 L, or about 2,000 L).

### Culture Systems

Some of the methods described herein use of a system (e.g., a perfusion culturing system) that includes a vessel and a first liquid medium disposed within the vessel (e.g., any of the culture media described herein). The vessel can have an internal volume (capacity) of between about 10 L to about 25,000 L (e.g., or any of the subranges of this range described herein). The vessel can be made from metal (e.g., stainless steel), plastic, or glass, or any combination thereof.

The system can include a device for agitating the liquid medium disposed within the vessel (e.g., an impellor and a motor that rotates the impellor). The vessel can include one or more ports (and optionally pumps) that allow for the addition of a material (e.g., a liquid medium, poloxamer-188, base or acid (as needed to regulate pH)) to the liquid medium disposed in the vessel and/or the removal of liquid medium (e.g., a clarified liquid medium or a sample of cell culture).

The culturing system can also include one or more sensors for monitoring the pH, dO₂, temperature, and pCO₂ in the liquid medium during a culturing period. In some embodiments, the culturing system can include one or more sensors including a capacitance sensor, a conductivity sensor, and/or a Raman sensor. In some embodiments, the bioreactor can include one or more sensors including a capacitance sensor, a conductivity sensor, and/or a Raman sensor. In some embodiments, the filter permeate can include one or more sensors including a capacitance sensor, a conductivity sensor, and/or a Raman sensor. The culturing system can include a filtration device (e.g., a device that performs alternating tangential filtration or tangential flow filtration) that processes a volume of cell culture to provide a clarified liquid medium that is substantially free of cells. The culturing system can include a heating/cooling device that allows for regulation of the temperature of the liquid medium disposed in the vessel. Additional features of cell culture systems are well known in the art.

### Perfusion Culturing

In some examples, perfusion culturing includes, during a period of time, removing from a vessel (e.g., a bioreactor) a first volume of a first liquid culture medium, and adding to the bioreactor a second volume of a second liquid culture medium, wherein the first volume and the second volume are about equal.

In other examples, perfusion culturing includes removing from a vessel (e.g., a bioreactor) a first volume of a first liquid culture medium, and adding to the vessel a second volume of a second liquid culture medium and a third volume of a third liquid culture medium, wherein the first volume and the sum of the second and third volumes are about equal.

The mammalian cells are retained in the bioreactor by some cell retention device or through techniques, such as cell settling in a settling cone.

The removal and addition of media in perfusion culturing can be performed simultaneously or sequentially, or some combination of the two. Further, removal and addition can be performed continuously, such as at a rate that removes and replaces a volume of between 0.1% to 400%, between about 25% to 400%, between about 25% to about 300%, between about 25% to about 200%, between about 25% to about 100%, between about 25% to about 50%, between about 50% to about 400%, between about 50% to about 100%, between about 50% to about 200%, between about 50% to about 300%, between about 50% to about 400%, between about 100% to about 400%, between about 100% to about 300%, between about 100% to about 200%, or about 25%, about 50%, about 75%, about 100%, about 150%, about 200%, about 250%, about 300%, about 350% or about 400% of the volume of the bioreactor.

The first volume of the first liquid culture medium removed and the second volume of the second liquid culture medium added (and optionally, the third liquid culture medium added) can in some instances be held approximately the same over each 24-hour period. As is known in the art, the rate at which the first volume of the first liquid culture medium is removed (volume/unit of time) and the rate at which the second volume of the second liquid culture medium is added (volume/unit of time) (and optionally, the rate at which the third volume of the third liquid culture medium is added (volume/unit of time)) can be varied and depends on the conditions of the particular cell culture system.

The rate at which the first volume of the first liquid culture medium is removed (volume/unit of time) and the rate at which the second volume of the second liquid culture medium is added (volume/unit of time) (and optionally, the rate at which the third volume of the third liquid culture medium is added (volume/unit of time)) can be about the same or can be different.

Alternatively, the volume removed and added can change by gradually increasing over each 24-hour period. For example, the volume of the first liquid culture medium removed and the volume of the second liquid culture medium added (and optionally, the volume of the third liquid culture medium added) within each 24-hour period can be increased over the culturing period. The volume can be increased over the culturing period from a volume that is between 0.5% to about 20% of the bioreactor volume. The volume can be increased over the culturing period to about 25% to about 150% of the bioreactor capacity or the volume of the cell culture at the start of the culturing period.

In some examples of the methods described herein, after the first 48 to 96 hours of the culturing period, in each 24-hour period (within the culturing period), the first volume of the first liquid culture medium removed and the second volume of the second liquid culture medium added (and optionally, also the third volume of the third liquid culture medium added) is about 10% to about 95%, about 10% to about 20%, about 20% to about 30%, about 30% to about 40%, about 40% to about 50%, about 50% to about 60%, about 60% to about 70%, about 70% to about 80%, about 80% to about 90%, about 85% to about 95%, about 60% to about 80%, or about 70% of the volume of the cell culture at the start of the culturing period.

Skilled practitioners will appreciate that the first liquid culture medium and the second liquid culture medium can be the same type of media. In other instances, the first liquid culture medium and the second liquid culture medium can be different types of liquid culture medium or different concentrations of liquid culture medium. The second liquid culture medium may be more concentrated with respect to one or more media components, as compared to the first liquid culture medium and the third liquid culture medium.

The first volume of the first liquid culture medium can be removed by using any automated system. For example alternating tangential flow filtration may be used. Alternatively, the first volume of the first liquid culture medium can be removed by seeping or gravity flow of the first volume of the first liquid culture medium through a sterile membrane with a molecular weight cut-off that excludes the mammalian cell. Alternatively, the first volume of the first liquid culture medium can be removed by stopping or significantly decreasing the rate of agitation for a period of at least 1 minute, at least 2 minutes, 3 minutes, 4 minutes, 5 minutes, 10 minutes, 15 minutes, 20 minutes, 25 minutes, 30 minutes, 40 minutes, 50 minutes, or 1 hour, and removing or aspirating the first volume of the first liquid culture medium from the top of the bioreactor.

The second volume of the second liquid culture medium (and optionally the third volume of the third liquid culture medium) can be added to the first liquid culture medium by a pump. The second liquid culture medium (and optionally the third liquid culture medium) can be added to the first liquid culture medium by pipetting or injecting the second volume of the second liquid culture medium (and optionally the third volume of the third liquid culture medium) directly onto the first liquid culture medium or in an automated fashion.

In some embodiments of the methods described herein include incubating the vessel with agitation and for period of time of at least about 7 days at a temperature between about 32° C. to about 39° C.; and during the period of time, continuously or periodically removing a first volume of the first liquid culture medium and adding to the first liquid culture medium a second volume of a second liquid culture medium, wherein the first and second volumes are about equal and the osmolality of the first and second liquid culture medium in the vessel is maintained at about 270 mOsm/kg to about 380 mOsm/kg over the period of time.

In some embodiments of the methods described herein include incubating the vessel with agitation and for period of time of at least about 7 days at a temperature between about 32° C. to about 39° C.; and during the period of time, continuously or periodically removing a first volume of the first liquid culture medium and adding to the first liquid culture medium a second volume of a second liquid culture medium and a third volume of a third liquid culture medium, where the first volume and the sum of the second and third volumes are about equal and the osmolality of the first, second, and third liquid culture medium in the vessel is maintained at about 270 mOsm/kg to about 380 mOsm/kg over the period of time.

Some examples of perfusion culturing include incubating a culturing system containing a first liquid culture medium with agitation and for a culturing period of at least about 7 days at a temperature between about 32° C. to about 39° C.; and continuously or periodically removing a first volume of the first liquid culture medium and adding to the first liquid culture medium a second volume of a second liquid culture medium, wherein the first and second volumes are about equal and the osmolality of the first and second liquid culture medium in the vessel is maintained at about 270 mOsm/kg to about 380 mOsm/kg over the period of time.

Some examples of perfusion culturing include incubating a culturing system containing a first liquid culture medium with agitation and for a culturing period of at least about 7 days at a temperature between about 32° C. to about 39° C.; and continuously or periodically removing a first volume of the first liquid culture medium and adding to the first liquid culture medium a second volume of a second liquid culture medium and a third volume of a third liquid culture medium, wherein the first volume and the sum of the second and third volumes are about equal and the osmolality of the first, second, and third liquid culture medium in the vessel is maintained at about 270 mOsm/kg to about 380 mOsm/kg over the period of time.

In some embodiments, a first liquid culture medium can have an osmolality of about 260 mOsm/kg to about 400 mOsm/kg (e.g., about 260 mOsm/kg to about 380 mOsm/kg, about 260 mOsm/kg to about 360 mOsm/kg, about 260 mOsm/kg to about 350 mOsm/kg, about 260 mOsm/kg to about 340 mOsm/kg, about 260 mOsm/kg to about 320 mOsm/kg, about 260 mOsm/kg to about 300 mOsm/kg, about 280 mOsm/kg to about 400 mOsm/kg, about 270 mOsm/kg to about 400 mOsm/kg, about 270 mOsm/kg to about 380 mOsm/kg, about 270 mOsm/kg to about 360 mOsm/kg, about 270 mOsm/kg to about 350 mOsm/kg, about 270 mOsm/kg to about 340 mOsm/kg, about 270 mOsm/kg to about 320 mOsm/kg, about 270 mOsm/kg to about 300 mOsm/kg, about 280 mOsm/kg to about 380 mOsm/kg, about 280 mOsm/kg to about 360 mOsm/kg, about 280 mOsm/kg to about 350 mOsm/kg, about 280 mOsm/kg to about 340 mOsm/kg, about 280 mOsm/kg to about 320 mOsm/kg, about 280 mOsm/kg to about 300 mOsm/kg, about 290 mOsm/kg to about 400 mOsm/kg, about 290 mOsm/kg to about 380 mOsm/kg, about 290 mOsm/kg to about 360 mOsm/kg, about 290 mOsm/kg to about 350 mOsm/kg, about 290 mOsm/kg to about 340 mOsm/kg, about 290 mOsm/kg to about 320 mOsm/kg, about 290 mOsm/kg to about 300 mOsm/kg, about 300 mOsm/kg to about 400 mOsm/kg, about 300 mOsm/kg to about 380 mOsm/kg, about 300 mOsm/kg to about 360 mOsm/kg, about 300 mOsm/kg to about 350 mOsm/kg, about 300 mOsm/kg to about 340 mOsm/kg, about 300 mOsm/kg to about 320 mOsm/kg, about 310 mOsm/kg to about 400 mOsm/kg, about 310 mOsm/kg to about 380 mOsm/kg, about 310 mOsm/kg to about 360 mOsm/kg, about 310 mOsm/kg to about 350 mOsm/kg, about 310 mOsm/kg to about 340 mOsm/kg, about 310 mOsm/kg to about 320 mOsm/kg, about 320 mOsm/kg to about 400 mOsm/kg, about 320 mOsm/kg to about 380 mOsm/kg, about 320 mOsm/kg to about 360 mOsm/kg, about 320 mOsm/kg to about 350 mOsm/kg, about 320 mOsm/kg to about 340 mOsm/kg, about 330 mOsm/kg to about 400 mOsm/kg, about 330 mOsm/kg to about 380 mOsm/kg, about 330 mOsm/kg to about 360 mOsm/kg, about 330 mOsm/kg to about 350 mOsm/kg, about 330 mOsm/kg to about 340 mOsm/kg, about 340 mOsm/kg to about 400 mOsm/kg, about 340 mOsm/kg to about 380 mOsm/kg, about 340 mOsm/kg to about 360 mOsm/kg, about 340 mOsm/kg to about 350 mOsm/kg, about 350 mOsm/kg to about 400 mOsm/kg, about 350 mOsm/kg to about 380 mOsm/kg, about 350 mOsm/kg to about 360 mOsm/kg, about 360 mOsm/kg to about 400 mOsm/kg, about 360 mOsm/kg to about 380 mOsm/kg, about 370 mOsm/kg to about 400 mOsm/kg, about 370 mOsm/kg to about 380 mOsm/kg, about 380 mOsm/kg to about 400 mOsm/kg, about 380 mOsm/kg to about 390 mOsm/kg, or about 390 mOsm/kg to about 400 mOsm/kg).

In some embodiments, the first liquid culture medium can have an osmolality of about 260 mOsm/kg, about 270 mOsm/kg, about 280 mOsm/kg, about 290 mOsm/kg, about 300 mOsm/kg, about 310 mOsm/kg, about 320 mOsm/kg, about 330 mOsm/kg, about 340 mOsm/kg, about 350 mOsm/kg, about 360 mOsm/kg, about 380 mOsm/kg, about 390 mOsm/kg, or about 400 mOsm/kg.

In some embodiments, the second liquid culture medium can have an osmolality of about 260 mOsm/kg to about 2,500 mOsm/kg (e.g., about 260 mOsm/kg to about 2,400 mOsm/kg, about 260 mOsm/kg to about 2,200 mOsm/kg, about 260 mOsm/kg to about 2,000 mOsm/kg, about 260 mOsm/kg to about 1,800 mOsm/kg, about 260 mOsm/kg to about 1,600 mOsm/kg, about 260 mOsm/kg to about 1,500 mOsm/kg, about 260 mOsm/kg to about 1,400 mOsm/kg, about 260 mOsm/kg to about 1,200 mOsm/kg, about 260 mOsm/kg to about 1,000 mOsm/kg, about 260 mOsm/kg to about 800 mOsm/kg, about 260 mOsm/kg to about 700 mOsm/kg, about 260 mOsm/kg to about 600 mOsm/kg, about 260 mOsm/kg to about 500 mOsm/kg, about 260 mOsm/kg to about 400 mOsm/kg, about 260 mOsm/kg to about 300 mOsm/kg, about 300 mOsm/kg to about 2,500 mOsm/kg, about 300 mOsm/kg to about 2,400 mOsm/kg, about 300 mOsm/kg to about 2,200 mOsm/kg, about 300 mOsm/kg to about 2,000 mOsm/kg, about 300 mOsm/kg to about 1,800 mOsm/kg, about 300 mOsm/kg to about 1,600 mOsm/kg, about 300 mOsm/kg to about 1,500 mOsm/kg, about 300 mOsm/kg to about 1,400 mOsm/kg, about 300 mOsm/kg to about 1,200 mOsm/kg, about 300 mOsm/kg to about 1,000 mOsm/kg, about 300 mOsm/kg to about 800 mOsm/kg, about 300 mOsm/kg to about 700 mOsm/kg, about 300 mOsm/kg to about 600 mOsm/kg, about 300 mOsm/kg to about 500 mOsm/kg, about 300 mOsm/kg to about 400 mOsm/kg, about 400 mOsm/kg to about 2,500 mOsm/kg, about 400 mOsm/kg to about 2,400 mOsm/kg, about 400 mOsm/kg to about 2,200 mOsm/kg, about 400 mOsm/kg to about 2,000 mOsm/kg, about 400 mOsm/kg to about 1,800 mOsm/kg, about 400 mOsm/kg to about 1,600 mOsm/kg, about 400 mOsm/kg to about 1,500 mOsm/kg, about 400 mOsm/kg to about 1,400 mOsm/kg, about 400 mOsm/kg to about 1,200 mOsm/kg, about 400 mOsm/kg to about 1,000 mOsm/kg, about 400 mOsm/kg to about 800 mOsm/kg, about 400 mOsm/kg to about 700 mOsm/kg, about 400 mOsm/kg to about 600 mOsm/kg, about 500 mOsm/kg to about 2,500 mOsm/kg, about 500 mOsm/kg to about 2,400 mOsm/kg, about 500 mOsm/kg to about 2,200 mOsm/kg, about 500 mOsm/kg to about 2,000 mOsm/kg, about 500 mOsm/kg to about 1,800 mOsm/kg, about 500 mOsm/kg to about 1,600 mOsm/kg, about 500 mOsm/kg to about 1,500 mOsm/kg, about 500 mOsm/kg to about 1,400 mOsm/kg, about 500 mOsm/kg to about 1,200 mOsm/kg, about 500 mOsm/kg to about 1,000 mOsm/kg, about 500 mOsm/kg to about 800 mOsm/kg, about 500 mOsm/kg to about 700 mOsm/kg, about 500 mOsm/kg to about 600 mOsm/kg, about 600 mOsm/kg to about 2,500 mOsm/kg, about 600 mOsm/kg to about 2,400 mOsm/kg, about 600 mOsm/kg to about 2,200 mOsm/kg, about 600 mOsm/kg to about 2,000 mOsm/kg, about 600 mOsm/kg to about 1,800 mOsm/kg, about 600 mOsm/kg to about 1,600 mOsm/kg, about 600 mOsm/kg to about 1,500 mOsm/kg, about 600 mOsm/kg to about 1,400 mOsm/kg, about 600 mOsm/kg to about 1,200 mOsm/kg, about 600 mOsm/kg to about 1,000 mOsm/kg, about 600 mOsm/kg to about 800 mOsm/kg, about 600 mOsm/kg to about 700 mOsm/kg, about 800 mOsm/kg to about 2,500 mOsm/kg, about 800 mOsm/kg to about 2400 mOsm/kg, about 800 mOsm/kg to about 2200 mOsm/kg, about 800 mOsm/kg to about 2,000 mOsm/kg, about 800 mOsm/kg to about 1,800 mOsm/kg, about 800 mOsm/kg to about 1,600 mOsm/kg, about 800 mOsm/kg to about 1,500 mOsm/kg, about 800 mOsm/kg to about 1,400 mOsm/kg, about 800 mOsm/kg to about 1,200 mOsm/kg, about 800 mOsm/kg to about 1,000 mOsm/kg, about 800 mOsm/kg to about 2,400 mOsm/kg, about 800 mOsm/kg to about 2,200 mOsm/kg, about 800 mOsm/kg to about 2,000 mOsm/kg, about 800 mOsm/kg to about 1,800 mOsm/kg, about 800 mOsm/kg to about 1,600 mOsm/kg, about 800 mOsm/kg to about 1,500 mOsm/kg, about 800 mOsm/kg to about 1,400 mOsm/kg, about 800 mOsm/kg to about 1,200 mOsm/kg, about 800 mOsm/kg to about 1,000 mOsm/kg, about 1,000 mOsm/kg to about 2,500 mOsm/kg, about 1,000 mOsm/kg to about 2,400 mOsm/kg, about 1,000 mOsm/kg to about 2,200 mOsm/kg, about 1,000 mOsm/kg to about 2,000 mOsm/kg, about 1,000 mOsm/kg to about 1,800 mOsm/kg, about 1,000 mOsm/kg to about 1,600 mOsm/kg, about 1,000 mOsm/kg to about 1,500 mOsm/kg, about 1,000 mOsm/kg to about 1,400 mOsm/kg, about 1,000 mOsm/kg to about 1,200 mOsm/kg, about 1,200 mOsm/kg to about 2,500 mOsm/kg, about 1,200 mOsm/kg to about 2,400 mOsm/kg, about 1,200 mOsm/kg to about 2,200 mOsm/kg, about 1,200 mOsm/kg to about 2,000 mOsm/kg, about 1,200 mOsm/kg to about 1,800 mOsm/kg, about 1,200 mOsm/kg to about 1,600 mOsm/kg, about 1,200 mOsm/kg to about 1,500 mOsm/kg, about 1,200 mOsm/kg to about 1,400 mOsm/kg, about 1,400 mOsm/kg to about 2,500 mOsm/kg, about 1,400 mOsm/kg to about 2,400 mOsm/kg, about 1,400 mOsm/kg to about 2,200 mOsm/kg, about 1,400 mOsm/kg to about 2,000 mOsm/kg, about 1,400 mOsm/kg to about 1,800 mOsm/kg, about 1,400 mOsm/kg to about 1,600 mOsm/kg, about 1,400 mOsm/kg to about 1,500 mOsm/kg, about 1,500 mOsm/kg to about 2,500 mOsm/kg, about 1,500 mOsm/kg to about 2,400 mOsm/kg, about 1,500 mOsm/kg to about 2,200 mOsm/kg, about 1,500 mOsm/kg to about 2,000 mOsm/kg, about 1,500 mOsm/kg to about 1,800 mOsm/kg, about 1,500 mOsm/kg to about 1,600 mOsm/kg, about 1,600 mOsm/kg to about 2,500 mOsm/kg, about 1,600 mOsm to about 2,400 mOsm/kg, about 1,600 mOsm/kg to about 2,200 mOsm/kg, about 1,600 mOsm/kg to about 2,000 mOsm/kg, about 1,800 mOsm/kg to about 2,500 mOsm/kg, about 1,800 mOsm/kg to about 2,400 mOsm/kg, about 1,800 mOsm/kg to about 2,200 mOsm/kg, about 1,800 mOsm/kg to about 2,000 mOsm/kg, about 2,000 mOsm/kg to about 2,500 mOsm/kg, about 2,000 mOsm/kg to about 2,400 mOsm/kg, about 2,000 mOsm/kg to about 2,200 mOsm/kg, about 2,200 mOsm/kg to about 2,500 mOsm/kg, about 2,200 mOsm/kg to about 2,400 mOsm/kg, or about 2,400 mOsm/kg to about 2,500 mOsm/kg).

In some embodiments, the second liquid culture medium has an osmolality of about 260 mOsm/kg, about 280 mOsm/kg, about 300 mOsm/kg, about 320 mOsm/kg, about 340 mOsm/kg, about 350 mOsm/kg, about 360 mOsm/kg, about 380 mOsm/kg, about 400 mOsm/kg, about 420 mOsm/kg, about 440 mOsm/kg, about 450 mOsm/kg, about 460 mOsm/kg, about 480 mOsm/kg, about 500 mOsm/kg, about 520 mOsm/kg, about 540 mOsm/kg, about 550 mOsm/kg, about 560 mOsm/kg, about 580 mOsm/kg, about 600 mOsm/kg, about 620 mOsm/kg, about 640 mOsm/kg, about 650 mOsm/kg, about 660 mOsm/kg, about 680 mOsm/kg, about 700 mOsm/kg, about 72 mOsm/kg, about 740 mOsm/kg, about 750 mOsm/kg, about 760 mOsm/kg, about 780 mOsm/kg, about 800 mOsm/kg, about 820 mOsm/kg, about 840 mOsm/kg, about 850 mOsm/kg, about 860 mOsm/kg, about 880 mOsm/kg, about 900 mOsm/kg, about 920 mOsm/kg, about 940 mOsm/kg, about 950 mOsm/kg, about 960 mOsm/kg, about 980 mOsm/kg, about 1000 mOsm/kg, about 1,100 mOsm/kg, about 1,200 mOsm/kg, about 1,300 mOsm/kg, about 1,400 mOsm/kg, about 1,500 mOsm/kg, about 1,600 mOsm/kg, about 1,700 mOsm/kg, about 1,800 mOsm/kg, about 1,900 mOsm/kg, about 2,000 mOsm/kg, about 2,100 mOsm/kg, about 2,200 mOsm/kg, about 2,300 mOsm/kg, about 2,400 mOsm/kg, or about 2,500 mOsm/kg.

Methods for determining the osmolality of a liquid culture medium are well known in the art. Osmality is measured by freezing point depression using off-line sampling. Commercially available osmometers are also well known in the art, e.g., OsmoTECH® pro multi-sample micro-osmometer. Continuous monitoring of osmolality can be performed through Raman spectroscopy. See, e.g., Moretto et al., Am Pharma Rev., 14(3), 2011, and Whelan et al., Biotechnol. Prog., 28(5): 1355-1362, September-October 2012.

Some embodiments of any of the methods described herein further include adding a third volume of an aqueous solution to the first and the second liquid culture medium in the vessel during the period of time, where the first volume and the sum of the second and third volumes are about equal and the osmolality of the first and second liquid culture medium, and the aqueous solution in the vessel is maintained at about 270 mOsm/kg to about 380 mOsm/kg (e.g., or any of the subranges of this range described herein) over the period of time. For example, the aqueous solution can be a salt solution, e.g., a sodium chloride solution and the potassium chloride solution. In some embodiments, the first liquid culture medium can be any of the first liquid culture medium described herein, and the second liquid culture medium can be any of the second liquid culture medium described herein.

Some embodiments of any of the methods described herein further include continuously or periodically adding to the first liquid culture medium a third volume of a third liquid culture medium, where the first volume and the sum of the second and third volumes are about equal and the osmolality of the first, second, and third liquid culture medium in the vessel is maintained at about 270 mOsm/kg to about 380 mOsm/kg (e.g., or any of the subranges of this range) over the period of time. In some embodiments of these methods, the first liquid culture medium can be any of the exemplary first liquid culture medium described herein, and the second liquid culture medium can be any of the exemplary second liquid culture media described herein. In some embodiments of these methods, maintaining the osmolality in the first, second, and third liquid culture medium in the vessel includes adjusting the flow rate of the second liquid culture medium during the period of time. In some embodiments, the adjusting step includes increasing the flow rate of the second liquid culture medium at some point during the period of time. In some examples, the adjusting step includes decreasing the flow rate of the second liquid culture medium at some point during the period of time.

In some examples, the third liquid culture medium can have an osmolality of about 0 mOsm/kg to about 12,000 mOsm/kg (e.g., about 0 mOsm/kg to about 11,000 mOsm/kg, about 0 mOsm/kg to about 10,000 mOsm/kg, about 0 mOsm/kg to about 9,000 mOsm/kg, about 0 mOsm/kg to about 8,000 mOsm/kg, about 0 mOsm/kg to about 7,000 mOsm/kg, about 0 mOsm/kg to about 6,000 mOsm/kg, about 0 mOsm/kg to about 5,000 mOsm/kg, about 0 mOsm/kg to about 4,000 mOsm/kg, about 0 mOsm/kg to about 3,000 mOsm/kg, about 0 mOsm/kg to about 2,000 mOsm/kg, about 0 mOsm/kg to about 1,000 mOsm/kg, about 0 mOsm/kg to about 800 mOsm/kg, about 0 mOsm/kg to about 600 mOsm/kg, about 0 mOsm/kg to about 500 mOsm/kg, about 0 mOsm/kg to about 400 mOsm/kg, about 0 mOsm/kg to about 350 mOsm/kg, about 0 mOsm/kg to about 300 mOsm/kg, about 0 mOsm/kg to about 250 mOsm/kg, about 0 mOsm/kg to about 200 mOsm/kg, about 0 mOsm/kg to about 150 mOsm/kg, about 0 mOsm/kg/to about 100 mOsm/kg, about 0 mOsm/kg to about 80 mOsm/kg, about 0 mOsm/kg to about 60 mOsm/kg, about 0 mOsm/kg, to about 40 mOsm/kg, about 0 mOsm/kg to about 20 mOsm/kg, about 0 mOsm/kg to about 10 mOsm/kg, about 0 mOsm/kg to about 5 mOsm/kg, about 0 mOsm/kg to about 1 mOsm/kg, about 1 mOsm/kg to about 12,000 mOsm/kg, about 1 mOsm/kg to about 11,000 mOsm/kg, about 1 mOsm/kg to about 10,000 mOsm/kg, about 1 mOsm/kg to about 9,000 mOsm/kg, about 1 mOsm/kg to about 8,000 mOsm/kg, about 1 mOsm/kg to about 7,000 mOsm/kg, about 1 mOsm/kg to about 6,000 mOsm/kg, about 1 mOsm/kg to about 5,000 mOsm/kg, about 1 mOsm/kg to about 4,000 mOsm/kg, about 1 mOsm/kg to about 3,000 mOsm/kg, about 1 mOsm/kg to about 2,000 mOsm/kg, about 1 mOsm/kg to about 1,000 mOsm/kg, about 1 mOsm/kg to about 800 mOsm/kg, about 1 mOsm/kg to about 600 mOsm/kg, about 1 mOsm/kg to about 500 mOsm/kg, about 1 mOsm/kg to about 400 mOsm/kg, about 1 mOsm/kg to about 350 mOsm/kg, about 1 mOsm/kg to about 300 mOsm/kg, about 1 mOsm/kg to about 250 mOsm/kg, about 1 mOsm/kg to about 200 mOsm/kg, about 1 mOsm/kg to about 150 mOsm/kg, about 1 mOsm/kg to about 100 mOsm/kg, about 1 mOsm/kg to about 80 mOsm/kg, about 1 mOsm/kg to about 60 mOsm/kg, about 1 mOsm/kg, to about 40 mOsm/kg, about 1 mOsm/kg to about 20 mOsm/kg, about 1 mOsm/kg to about 10 mOsm/kg, about 1 mOsm/kg to about 5 mOsm/kg, about 5 mOsm/kg to about 12,000 mOsm/kg, about 5 mOsm/kg to about 11,000 mOsm/kg, about 5 mOsm/kg to about 10,000 mOsm/kg, about 5 mOsm/kg to about 9,000 mOsm/kg, about 5 mOsm/kg to about 8,000 mOsm/kg, about 5 mOsm/kg to about 7,000 mOsm/kg, about 5 mOsm/kg to about 6,000 mOsm/kg, about 5 mOsm/kg to about 5,000 mOsm/kg, about 5 mOsm/kg to about 4,000 mOsm/kg, about 5 mOsm/kg to about 3,000 mOsm/kg, about 5 mOsm/kg to about 2,000 mOsm/kg, about 5 mOsm/kg to about 1,000 mOsm/kg, about 5 mOsm/kg to about 800 mOsm/kg, about 5 mOsm/kg to about 600 mOsm/kg, about 5 mOsm/kg to about 500 mOsm/kg, about 5 mOsm/kg to about 400 mOsm/kg, about 5 mOsm/kg to about 350 mOsm/kg, about 5 mOsm/kg to about 300 mOsm/kg, about 5 mOsm/kg to about 250 mOsm/kg, about 5 mOsm/kg to about 200 mOsm/kg, about 5 mOsm/kg to about 150 mOsm/kg, about 5 mOsm/kg to about 100 mOsm/kg, about 5 mOsm/kg to about 80 mOsm/kg, about 5 mOsm/kg to about 60 mOsm/kg, about 5 mOsm/kg, to about 40 mOsm/kg, about 5 mOsm/kg to about 20 mOsm/kg, about 5 mOsm/kg to about 10 mOsm/kg, about 10 mOsm/kg to about 12,000 mOsm/kg, about 10 mOsm/kg to about 11,000 mOsm/kg, about 10 mOsm/kg to about 10,000 mOsm/kg, about 10 mOsm/kg to about 9,000 mOsm/kg, about 10 mOsm/kg to about 8,000 mOsm/kg, about 10 mOsm/kg to about 7,000 mOsm/kg, about 10 mOsm/kg to about 6,000 mOsm/kg, about 10 mOsm/kg to about 5,000 mOsm/kg, about 10 mOsm/kg to about 4,000 mOsm/kg, about 10 mOsm/kg to about 3,000 mOsm/kg, about 10 mOsm/kg to about 2,000 mOsm/kg, about 10 mOsm/kg to about 1,000 mOsm/kg, about 10 mOsm/kg to about 800 mOsm/kg, about 10 mOsm/kg to about 600 mOsm/kg, about 10 mOsm/kg to about 500 mOsm/kg, about 10 mOsm/kg to about 400 mOsm/kg, about 10 mOsm/kg to about 350 mOsm/kg, about 10 mOsm/kg to about 300 mOsm/kg, about 10 mOsm/kg to about 250 mOsm/kg, about 10 mOsm/kg to about 200 mOsm/kg, about 10 mOsm/kg to about 150 mOsm/kg, about 10 mOsm/kg to about 100 mOsm/kg, about 10 mOsm/kg to about 80 mOsm/kg, about 10 mOsm/kg to about 60 mOsm/kg, about 10 mOsm/kg, to about 40 mOsm/kg, about 10 mOsm/kg to about 20 mOsm/kg, about 20 mOsm/kg to about 12,000 mOsm/kg, about 20 mOsm/kg to about 11,000 mOsm/kg, about 20 mOsm/kg to about 10,000 mOsm/kg, about 20 mOsm/kg to about 9,000 mOsm/kg, about 20 mOsm/kg to about 8,000 mOsm/kg, about 20 mOsm/kg to about 7,000 mOsm/kg, about 20 mOsm/kg to about 6,000 mOsm/kg, about 20 mOsm/kg to about 5,000 mOsm/kg, about 20 mOsm/kg to about 4,000 mOsm/kg, about 20 mOsm/kg to about 3,000 mOsm/kg, about 20 mOsm/kg to about 2,000 mOsm/kg, about 20 mOsm/kg to about 1,000 mOsm/kg, about 20 mOsm/kg to about 800 mOsm/kg, about 20 mOsm/kg to about 600 mOsm/kg, about 20 mOsm/kg to about 500 mOsm/kg, about 20 mOsm/kg to about 400 mOsm/kg, about 20 mOsm/kg to about 350 mOsm/kg, about 20 mOsm/kg to about 300 mOsm/kg, about 20 mOsm/mg to about 250 mOsm/kg, about 20 mOsm/kg to about 200 mOsm/kg, about 20 mOsm/kg to about 150 mOsm/kg, about 20 mOsm/kg to about 100 mOsm/kg, about 20 mOsm/kg to about 80 mOsm/kg, about 20 mOsm/kg to about 60 mOsm/kg, about 20 mOsm/kg, to about 40 mOsm/kg, about 40 mOsm/kg to about 12,000 mOsm/kg, about 40 mOsm/kg to about 11,000 mOsm/kg, about 40 mOsm/kg to about 10,000 mOsm/kg, about 40 mOsm/kg to about 9,000 mOsm/kg, about 40 mOsm/kg to about 8,000 mOsm/kg, about 40 mOsm/kg to about 7,000 mOsm/kg, about 40 mOsm/kg to about 6,000 mOsm/kg, about 40 mOsm/kg to about 5,000 mOsm/kg, about 40 mOsm/kg to about 4,000 mOsm/kg, about 40 mOsm/kg to about 3,000 mOsm/kg, about 40 mOsm/kg to about 2,000 mOsm/kg, about 40 mOsm/kg to about 1,000 mOsm/kg, about 40 mOsm/kg to about 800 mOsm/kg, about 40 mOsm/kg to about 600 mOsm/kg, about 40 mOsm/kg to about 500 mOsm/kg, about 40 mOsm/kg to about 400 mOsm/kg, about 40 mOsm/kg to about 350 mOsm/kg, about 40 mOsm/kg to about 300 mOsm/kg, about 40 mOsm/kg to about 250 mOsm/kg, about 40 mOsm/kg to about 200 mOsm/kg, about 40 mOsm/kg to about 150 mOsm/kg, about 40 mOsm/kg to about 100 mOsm/kg, about 40 mOsm/kg to about 80 mOsm/kg, about 40 mOsm/kg to about 60 mOsm/kg, about 60 mOsm/kg to about 12,000 mOsm/kg, about 60 mOsm/kg to about 11,000 mOsm/kg, about 60 mOsm/kg to about 10,000 mOsm/kg, about 60 mOsm/kg to about 9,000 mOsm/kg, about 60 mOsm/kg to about 8,000 mOsm/kg, about 60 mOsm/kg to about 7,000 mOsm/kg, about 60 mOsm/kg to about 6,000 mOsm/kg, about 60 mOsm/kg to about 5,000 mOsm/kg, about 60 mOsm/kg to about 4,000 mOsm/kg, about 60 mOsm/kg to about 3,000 mOsm/kg, about 60 mOsm/kg to about 2,000 mOsm/kg, about 60 mOsm/kg to about 1,000 mOsm/kg, about 60 mOsm/kg to about 800 mOsm/kg, about 60 mOsm/kg to about 600 mOsm/kg, about 60 mOsm/kg to about 500 mOsm/kg, about 60 mOsm/kg to about 400 mOsm/kg, about 60 mOsm/kg to about 350 mOsm/kg, about 60 mOsm/kg to about 300 mOsm/kg, about 60 mOsm/kg to about 250 mOsm/kg, about 60 mOsm/kg to about 200 mOsm/kg, about 60 mOsm/kg to about 150 mOsm/kg, about 60 mOsm/kg to about 100 mOsm/kg, about 60 mOsm/kg to about 80 mOsm/kg, about 80 mOsm/kg to about 12,000 mOsm/kg, about 80 mOsm/kg to about 11,000 mOsm/kg, about 80 mOsm/kg to about 10,000 mOsm/kg, about 80 mOsm/kg to about 9,000 mOsm/kg, about 80 mOsm/kg to about 8,000 mOsm/kg, about 80 mOsm/kg to about 7,000 mOsm/kg, about 80 mOsm/kg to about 6,000 mOsm/kg, about 80 mOsm/kg to about 5,000 mOsm/kg, about 80 mOsm/kg to about 4,000 mOsm/kg, about 80 mOsm/kg to about 3,000 mOsm/kg, about 80 mOsm/kg to about 2,000 mOsm/kg, about 80 mOsm/kg to about 1,000 mOsm/kg, about 80 mOsm/kg to about 800 mOsm/kg, about 80 mOsm/kg to about 600 mOsm/kg, about 80 mOsm/kg to about 500 mOsm/kg, about 80 mOsm/kg to about 400 mOsm/kg, about 80 mOsm/kg to about 350 mOsm/kg, about 80 mOsm/kg to about 300 mOsm/kg, about 80 mOsm/kg to about 250 mOsm/kg, about 80 mOsm/kg to about 200 mOsm/kg, about 80 mOsm/kg to about 150 mOsm/kg, about 80 mOsm/kg to about 100 mOsm/kg, about 100 mOsm/kg to about 12,000 mOsm/kg, about 100 mOsm/kg to about 11,000 mOsm/kg, about 100 mOsm/kg to about 10,000 mOsm/kg, about 100 mOsm/kg to about 9,000 mOsm/kg, about 100 mOsm/kg to about 8,000 mOsm/kg, about 100 mOsm/kg to about 7,000 mOsm/kg, about 100 mOsm/kg to about 6,000 mOsm/kg, about 100 mOsm/kg to about 5,000 mOsm/kg, about 100 mOsm/kg to about 4,000 mOsm/kg, about 100 mOsm/kg to about 3,000 mOsm/kg, about 100 mOsm/kg to about 2,000 mOsm/kg, about 100 mOsm/kg to about 1,000 mOsm/kg, about 100 mOsm/kg to about 800 mOsm/kg, about 100 mOsm/kg to about 600 mOsm/kg, about 100 mOsm/kg to about 500 mOsm/kg, about 100 mOsm/kg to about 400 mOsm/kg, about 100 mOsm/kg to about 350 mOsm/kg, about 100 mOsm/kg to about 300 mOsm/kg, about 100 mOsm/kg to about 250 mOsm/kg, about 100 mOsm/kg to about 200 mOsm/kg, about 100 mOsm/kg to about 150 mOsm/kg, about 150 mOsm/kg to about 12,000 mOsm/kg, about 150 mOsm/kg to about 11,000 mOsm/kg, about 150 mOsm/kg to about 10,000 mOsm/kg, about 150 mOsm/kg to about 9,000 mOsm/kg, about 150 mOsm/kg to about 8,000 mOsm/kg, about 150 mOsm/kg to about 7,000 mOsm/kg, about 150 mOsm/kg to about 6,000 mOsm/kg, about 150 mOsm/kg to about 5,000 mOsm/kg, about 150 mOsm/kg to about 4,000 mOsm/kg, about 150 mOsm/kg to about 3,000 mOsm/kg, about 150 mOsm/kg to about 2,000 mOsm/kg, about 150 mOsm/kg to about 1,000 mOsm/kg, about 150 mOsm/kg to about 800 mOsm/kg, about 150 mOsm/kg to about 600 mOsm/kg, about 150 mOsm/kg to about 500 mOsm/kg, about 150 mOsm/kg to about 400 mOsm/kg, about 150 mOsm/kg to about 350 mOsm/kg, about 150 mOsm/kg to about 300 mOsm/kg, about 150 mOsm/kg to about 250 mOsm/kg, about 150 mOsm/kg to about 200 mOsm/kg, about 200 mOsm/kg to about 12,000 mOsm/kg, about 200 mOsm/kg to about 11,000 mOsm/kg, about 200 mOsm/kg to about 10,000 mOsm/kg, about 200 mOsm/kg to about 9,000 mOsm/kg, about 200 mOsm/kg to about 8,000 mOsm/kg, about 200 mOsm/kg to about 7,000 mOsm/kg, about 200 mOsm/kg to about 6,000 mOsm/kg, about 200 mOsm/kg to about 5,000 mOsm/kg, about 200 mOsm/kg to about 4,000 mOsm/kg, about 200 mOsm/kg to about 3,000 mOsm/kg, about 200 mOsm/kg to about 2,000 mOsm/kg, about 200 mOsm/kg to about 1,000 mOsm/kg, about 200 mOsm/kg to about 800 mOsm/kg, about 200 mOsm/kg to about 600 mOsm/kg, about 200 mOsm/kg to about 500 mOsm/kg, about 200 mOsm/kg to about 400 mOsm/kg, about 200 mOsm/kg to about 350 mOsm/kg, about 200 mOsm/kg to about 300 mOsm/kg, about 200 mOsm/kg to about 250 mOsm/kg, about 250 mOsm/kg to about 12,000 mOsm/kg, about 250 mOsm/kg to about 11,000 mOsm/kg, about 250 mOsm/kg to about 10,000 mOsm/kg, about 250 mOsm/kg to about 9,000 mOsm/kg, about 250 mOsm/kg to about 8,000 mOsm/kg, about 250 mOsm/kg to about 7,000 mOsm/kg, about 250 mOsm/kg to about 6,000 mOsm/kg, about 250 mOsm/kg to about 5,000 mOsm/kg, about 250 mOsm/kg to about 4,000 mOsm/kg, about 250 mOsm/kg to about 3,000 mOsm/kg, about 250 mOsm/kg to about 2,000 mOsm/kg, about 250 mOsm/kg to about 1,000 mOsm/kg, about 250 mOsm/kg to about 800 mOsm/kg, about 250 mOsm/kg to about 600 mOsm/kg, about 250 mOsm/kg to about 500 mOsm/kg, about 250 mOsm/kg to about 400 mOsm/kg, about 250 mOsm/kg to about 350 mOsm/kg, about 250 mOsm/kg to about 300 mOsm/kg, about 300 mOsm/kg to about 12,000 mOsm/kg, about 300 mOsm/kg to about 11,000 mOsm/kg, about 300 mOsm/kg to about 10,000 mOsm/kg, about 300 mOsm/kg to about 9,000 mOsm/kg, about 300 mOsm/kg to about 8,000 mOsm/kg, about 300 mOsm/kg to about 7,000 mOsm/kg, about 300 mOsm/kg to about 6,000 mOsm/kg, about 300 mOsm/kg to about 5,000 mOsm/kg, about 300 mOsm/kg to about 4,000 mOsm/kg, about 300 mOsm/kg to about 3,000 mOsm/kg, about 300 mOsm/kg to about 2,000 mOsm/kg, about 300 mOsm/kg to about 1,000 mOsm/kg, about 300 mOsm/kg to about 800 mOsm/kg, about 300 mOsm/kg to about 600 mOsm/kg, about 300 mOsm/kg to about 500 mOsm/kg, about 300 mOsm/kg to about 400 mOsm/kg, about 300 mOsm/kg to about 350 mOsm/kg, about 350 mOsm/kg to about 12,000 mOsm/kg, about 350 mOsm/kg to about 11,000 mOsm/kg, about 350 mOsm/kg to about 10,000 mOsm/kg, about 350 mOsm/kg to about 9,000 mOsm/kg, about 350 mOsm/kg to about 8,000 mOsm/kg, about 350 mOsm/kg to about 7,000 mOsm/kg, about 350 mOsm/kg to about 6,000 mOsm/kg, about 350 mOsm/kg to about 5,000 mOsm/kg, about 350 mOsm/kg to about 4,000 mOsm/kg, about 350 mOsm/kg to about 3,000 mOsm/kg, about 350 mOsm/kg to about 2,000 mOsm/kg, about 350 mOsm/kg to about 1,000 mOsm/kg, about 350 mOsm/kg to about 800 mOsm/kg, about 350 mOsm/kg to about 600 mOsm/kg, about 350 mOsm/kg to about 500 mOsm/kg, about 350 mOsm/kg to about 400 mOsm/kg, about 400 mOsm/kg to about 12,000 mOsm/kg, about 400 mOsm/kg to about 11,000 mOsm/kg, about 400 mOsm/kg to about 10,000 mOsm/kg, about 400 mOsm/kg to about 9,000 mOsm/kg, about 400 mOsm/kg to about 8,000 mOsm/kg, about 400 mOsm/kg to about 7,000 mOsm/kg, about 400 mOsm/kg to about 6,000 mOsm/kg, about 400 mOsm/kg to about 5,000 mOsm/kg, about 400 mOsm/kg to about 4,000 mOsm/kg, about 400 mOsm/kg to about 3,000 mOsm/kg, about 400 mOsm/kg to about 2,000 mOsm/kg, about 400 mOsm/kg to about 1,000 mOsm/kg, about 400 mOsm/kg to about 800 mOsm/kg, about 400 mOsm/kg to about 600 mOsm/kg, about 400 mOsm/kg to about 500 mOsm/kg, about 500 mOsm/kg to about 12,000 mOsm/kg, about 500 mOsm/kg to about 11,000 mOsm/kg, about 500 mOsm/kg to about 10,000 mOsm/kg, about 500 mOsm/kg to about 9,000 mOsm/kg, about 500 mOsm/kg to about 8,000 mOsm/kg, about 500 mOsm/kg to about 7,000 mOsm/kg, about 500 mOsm/kg to about 6,000 mOsm/kg, about 500 mOsm/kg to about 5,000 mOsm/kg, about 500 mOsm/kg to about 4,000 mOsm/kg, about 500 mOsm/kg to about 3,000 mOsm/kg, about 500 mOsm/kg to about 2,000 mOsm/kg, about 500 mOsm/kg to about 1,000 mOsm/kg, about 500 mOsm/kg to about 800 mOsm/kg, about 500 mOsm/kg to about 600 mOsm/kg, about 600 mOsm/kg to about 12,000 mOsm/kg, about 600 mOsm/kg to about 11,000 mOsm/kg, about 600 mOsm/kg to about 10,000 mOsm/kg, about 600 mOsm/kg to about 9,000 mOsm/kg, about 600 mOsm/kg to about 8,000 mOsm/kg, about 600 mOsm/kg to about 7,000 mOsm/kg, about 600 mOsm/kg to about 6,000 mOsm/kg, about 600 mOsm/kg to about 5,000 mOsm/kg, about 600 mOsm/kg to about 4,000 mOsm/kg, about 600 mOsm/kg to about 3,000 mOsm/kg, about 600 mOsm/kg to about 2,000 mOsm/kg, about 600 mOsm/kg to about 1,000 mOsm/kg, about 600 mOsm/kg to about 800 mOsm/kg, about 800 mOsm/kg to about 12,000 mOsm/kg, about 800 mOsm/kg to about 11,000 mOsm/kg, about 800 mOsm/kg to about 10,000 mOsm/kg, about 800 mOsm/kg to about 9,000 mOsm/kg, about 800 mOsm/kg to about 8,000 mOsm/kg, about 800 mOsm/kg to about 7,000 mOsm/kg, about 800 mOsm/kg to about 6,000 mOsm/kg, about 800 mOsm/kg to about 5,000 mOsm/kg, about 800 mOsm/kg to about 4,000 mOsm/kg, about 800 mOsm/kg to about 3,000 mOsm/kg, about 800 mOsm/kg to about 2,000 mOsm/kg, about 800 mOsm/kg to about 1,000 mOsm/kg, about 1,000 mOsm/kg to about 12,000 mOsm/kg, about 1,000 mOsm/kg to about 11,000 mOsm/kg, about 1,000 mOsm/kg to about 10,000 mOsm/kg, about 1,000 mOsm/kg to about 9,000 mOsm/kg, about 1,000 mOsm/kg to about 8,000 mOsm/kg, about 1,000 mOsm/kg to about 7,000 mOsm/kg, about 1,000 mOsm/kg to about 6,000 mOsm/kg, about 1,000 mOsm/kg to about 5,000 mOsm/kg, about 1,000 mOsm/kg to about 4,000 mOsm/kg, about 1,000 mOsm/kg to about 3,000 mOsm/kg, about 1,000 mOsm/kg to about 2,000 mOsm/kg, about 2,000 mOsm/kg to about 12,000 mOsm/kg, about 2,000 mOsm/kg to about 11,000 mOsm/kg, about 2,000 mOsm/kg to about 10,000 mOsm/kg, about 2,000 mOsm/kg to about 9,000 mOsm/kg, about 2,000 mOsm/kg to about 8,000 mOsm/kg, about 2,000 mOsm/kg to about 7,000 mOsm/kg, about 2,000 mOsm/kg to about 6,000 mOsm/kg, about 2,000 mOsm/kg to about 5,000 mOsm/kg, about 2,000 mOsm/kg to about 4,000 mOsm/kg, about 2,000 mOsm/kg to about 3,000 mOsm/kg, about 3,000 mOsm/kg to about 12,000 mOsm/kg, about 3,000 mOsm/kg to about 11,000 mOsm/kg, about 3,000 mOsm/kg to about 10,000 mOsm/kg, about 3,000 mOsm/kg to about 9,000 mOsm/kg, about 3,000 mOsm/kg to about 8,000 mOsm/kg, about 3,000 mOsm/kg to about 7,000 mOsm/kg, about 3,000 mOsm/kg to about 6,000 mOsm/kg, about 3,000 mOsm/kg to about 5,000 mOsm/kg, about 3,000 mOsm/kg to about 4,000 mOsm/kg, about 4,000 mOsm/kg to about 12,000 mOsm/kg, about 4,000 mOsm/kg to about 11,000 mOsm/kg, about 4,000 mOsm/kg to about 10,000 mOsm/kg, about 4,000 mOsm/kg to about 9,000 mOsm/kg, about 4,000 mOsm/kg to about 8,000 mOsm/kg, about 4,000 mOsm/kg to about 7,000 mOsm/kg, about 4,000 mOsm/kg to about 6,000 mOsm/kg, about 4,000 mOsm/kg to about 5,000 mOsm/kg, about 5,000 mOsm/kg to about 12,000 mOsm/kg, about 5,000 mOsm/kg to about 11,000 mOsm/kg, about 5,000 mOsm/kg to about 10,000 mOsm/kg, about 5,000 mOsm/kg to about 9,000 mOsm/kg, about 5,000 mOsm/kg to about 8,000 mOsm/kg, about 5,000 mOsm/kg to about 7,000 mOsm/kg, about 5,000 mOsm/kg to about 5,000 mOsm/kg, about 6,000 mOsm/kg to about 12,000 mOsm/kg, about 6,000 mOsm/kg to about 11,000 mOsm/kg, about 6,000 mOsm/kg to about 10,000 mOsm/kg, about 6,000 mOsm/kg to about 9,000 mOsm/kg, about 6,000 mOsm/kg to about 8,000 mOsm/kg, about 6,000 mOsm/kg to about 7,000 mOsm/kg, about 7,000 mOsm/kg to about 12,000 mOsm/kg, about 7,000 mOsm/kg to about 11,000 mOsm/kg, about 7,000 mOsm/kg to about 10,000 mOsm/kg, about 7,000 mOsm/kg to about 9,000 mOsm/kg, about 7,000 mOsm/kg to about 8,000 mOsm/kg, about 8,000 mOsm/kg to about 12,000 mOsm/kg, about 8,000 mOsm/kg to about 11,000 mOsm/kg, about 8,000 mOsm/kg to about 10,000 mOsm/kg, about 8,000 mOsm/kg to about 9,000 mOsm/kg, about 9,000 mOsm/kg to about 12,000 mOsm/kg, about 9,000 mOsm/kg to about 11,000 mOsm/kg, about 9,000 mOsm/kg to about 10,000 mOsm/kg, about 10,000 mOsm/kg to about 12,000 mOsm/kg, or about 11,000 mOsm/kg to about 12,000 mOsm/kg). In some embodiments, the third liquid culture medium includes sodium chloride (NaCl) or potassium chloride (KCl).

Some embodiments further include adjusting one or both of the pH and the pCO₂ of the first, second, and third liquid culture medium in the vessel at some point during the period of time. In some examples, the method includes increasing the pCO₂ and increasing the pH of the first, second, and third liquid culture medium in the vessel over the period of time. In some examples, the pCO₂ and the pH of the first, second, and third liquid culture medium are measured in the vessel over the period of time.

In some embodiments of any of the methods described herein, the osmolality of the first and/or the second liquid culture medium is controlled by one or more of: the ratio of the first and/or the second liquid culture medium and an aqueous solution (e.g., water), the ratio of the first and/or the second liquid culture medium and a concentration osmolite (e.g., sodium chloride), the viable cell density in the vessel (e.g., any of the vessels described herein (e.g., a bioreactor)), the pH of the first and/or second liquid culture medium, the level of dissolved CO₂ (dCO₂) in the vessel (e.g., any of the vessels described herein (e.g., a bioreactor)), the carbon (e.g., glucose) input into the vessel (e.g., any of the vessels described herein (e.g., a bioreactor)).

### Pluronic® F-68 (Poloxamer-188)

Poloxamer-188 is a molecule known in the art. Poloxamer-188 is a non-ionic synthetic block copolymer of ethylene oxide and propylene oxide. Poloxamer-188 has an average molecular weight of between 7680 to about 9510, and about 81.8% 1.9% of its compositions by weight is represented by oxyethylene. For example, poloxamer-188 has the structure shown in Formula I below, where a is 80 and b is 27. Poloxamer-188 has a CAS number of 9003-11-6.

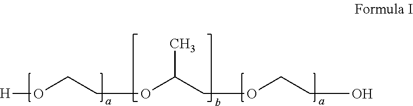

Poloxamer-188 is commercially available from a number of different vendors including, e.g., Sigma-Aldrich (St. Louis, Mo.), Mediatech, Inc. (Manassas, Va.), MAST Therapeutics, Inc. (San Diego, Calif.), EMD Millipore (Billerica, Mass.), BASF (Ludwigshafen, Germany), Pluronic® F-68 Life Technologies (Carlsbad, Calif.), and PhytoTechnology Laboratories, LLC (Shawnee Mission, Kans.).

In some embodiments, the second liquid culture medium can include a concentration of greater than about 8 g/L (e.g., a concentration of about 9.0 g/L or a concentration greater than 9.0 g/L, a concentration of about 10.0 g/L or a concentration greater than 10.0 g/L, a concentration of about 12.0 g/L or a concentration greater than 12.0 g/L, a concentration of about 14.0 g/L or a concentration greater than 14.0 g/L, or a concentration of about 16.0 g/L or a concentration greater than 16.0 g/L).

In some embodiments, the second liquid culture medium can include poloxamer-188 at a concentration of between about 8 g/L and about 16 g/L (e.g., between about 8.0 g/L and about 14.0 g/L, between about 8.0 g/L and about 12.0 g/L, between about 8.0 g/L and about 10.0 g/L, between about 10.0 g/L and about 16.0 g/L, between about 10.0 g/L and about 12.0 g/L, between about 12.0 g/L and about 16.0 g/L, between about 12.0 g/L and about 14.0 g/L, between about 14.0 g/L and about 16.0 g/L, or between about 15.0 g/L and about 16.0 g/L).

In some embodiments, the third liquid culture medium can include poloxamer-188 at a concentration of between about 0 g/L and about 8 g/L (e.g., between about 0 g/L and about 6 g/L, about 0 g/L to about 4 g/L, about 0 g/L to about 2 g/L, about 0 g/L to about 1 g/L, between about 0.5 g/L and about 8 g/L, between about 0.5 g/L and about 6.0 g/L, between about 0.5 g/L and about 4.0 g/L, between about 0.5 g/L and about 2.0 g/L, between about 1.0 g/L and about 8 g/L, between about 1.0 g/L and about 6.0 g/L, between about 1.0 g/L and about 4.0 g/L, between about 2.0 g/L and about 8 g/L, between about 2.0 g/L and about 6.0 g/L, between about 2.0 g/L and about 4.0 g/L, between about 5.0 g/L and about 8 g/L, between about 5.0 g/L and about 6.0 g/L, between about 6.0 g/L and about 8 g/L, or between about 7.0 g/L and about 8 g/L).

Some embodiments of any of the methods provided herein include increasing the poloxamer-188 concentration in the culture over time. In some embodiments, the medium includes a poloxamer-188 concentration that is selected based on one or more factors selected from the group of: gas flow rate and viable cell density in the medium. In these methods, the selected poloxamer-188 concentration in the medium can be achieved by adding poloxamer-188 to the medium prior to the culturing step and/or by adding poloxamer-188 to the medium during the culturing step. The selected poloxamer-188 concentration can be any of the exemplary concentrations or ranges of poloxamer-188 described herein.

### Culture Media

Liquid culture media are known in the art. The liquid culture media (e.g., a first, second, and/or third liquid culture medium) can be supplemented with a mammalian serum (e.g., fetal calf serum and bovine serum), and/or a growth hormone or growth factor (e.g., insulin, transferrin, and epidermal growth factor). Alternatively or in addition, the liquid culture media (e.g., a first, second, and/or third liquid medium) can be a chemically-defined liquid culture medium, an animal-derived component free liquid culture medium, a serum-free liquid culture medium, or a serum-containing liquid culture medium. Non-limiting examples of chemically-defined liquid culture media, animal-derived component free liquid culture media, serum-free liquid culture media, and serum-containing liquid culture media are commercially available.

A liquid medium typically contains an energy source (e.g., a carbohydrate, such as glucose), essential amino acids (e.g., the basic set of twenty amino acids plus cysteine), vitamins and/or other organic compounds required at low concentrations, free fatty acids, and/or trace elements. The liquid culture media (e.g., a first, second, and/or third liquid medium) can, if desired, be supplemented with, e.g., a mammalian hormone or growth factor (e.g., insulin, transferrin, or epidermal growth factor), salts and buffers (e.g., calcium, magnesium, and phosphate salts), nucleosides and bases (e.g., adenosine, thymidine, and hypoxanthine), protein and tissue hydrolysates, and/or any combination of these additives.

A wide variety of different liquid culture media that can be used to culture cells (e.g., mammalian cells) in any of the methods described herein are known in the art. Medium components that also may be useful in the present processes include, but are not limited to, chemically-defined (CD) hydrolysates, e.g., CD peptone, CD polypeptides (two or more amino acids), and CD growth factors. Additional examples of liquid medium and medium components are known in the art.

Skilled practitioners will appreciate that the first liquid culture medium, the second liquid culture medium, and the third liquid culture medium described herein can be the same type of media or different media.

Liquid culture medium obtained from a cell culture can be filtered or clarified to obtain a liquid culture medium that is substantially free of cells and/or viruses. Methods for filtering or clarifying a liquid culture medium in order to remove cells are known in the art (e.g., 0.2-μm filtration and filtration using an Alternating Tangential Flow (ATFTM) system). Mammalian cells can also be removed from liquid medium using centrifugation and removing the supernatant that is liquid culture medium that is substantially free of cells, or by allowing the cells to settle to the gravitational bottom of a vessel or bioreactor containing the liquid medium, and removing the liquid culture medium (the liquid culture medium that is substantially free of cells) that is distant from the settled recombinant cells.

In some embodiments of any of the methods described herein, the first, second, and/or third liquid culture medium has a pH of about 6.0 to about 6.8 (e.g., any of the subranges therein).

In some embodiments of any of the methods described herein, the first, second and/or third liquid culture medium includes sodium chloride or potassium chloride. In some embodiments of any of the methods described herein, the first, second and/or third liquid culture medium further includes one or more of: L-arginine, L-asparagine, L-glutamine, L-histidine, hydroxy-L-proline, L-isoleucine, L-leucine, L-lysine, L-methionine, L-phenylalanine, L-proline, L-serine, L-threonine, L-tryptophan, L-valine, choline, inositol, biotin, calcium, folic acid, niacinamide, pyridoxine, riboflavin, thiamine, vitamin B12, lipoic acid, glutathione, linoleic acid, pyruvate, HEPES, ferrous iron, citric acid, zinc, copper, selenite, ethanolamine, putrescine, spermine, potassium, phosphate, magnesium, aluminum, vanadate, molybdate, manganese, nickel, silicate, and tin.

pH (6-8, 6.5-7.5)

The methods described herein are performed at a pH of about 6.8 to about 7.1.

The first, second, and/or third liquid culture medium can have a pH of about 6.8 to about 7.1 (e.g., about 6.8 to about 7.0, about 6.8 to about 6.9, about 6.9 to about 7.1, about 6.9 to about 7.0, or about 7.0 to about 7.2).

In some embodiments, the first, second, and/or third liquid culture medium can have a pH of about 6.8, about 6.9, about 7.0, or about 7.1.

### Temperature (30° C.-40° C.; 37° C.)

The step of incubating the mammalian cell can be performed at a temperature of about 32° C. to about 39° C. (e.g., about 32° C. to about 38° C., about 32° C. to about 36° C., about 32° C. to about 35° C., about 32° C. to about 34° C., about 34° C. to about 39° C., about 34° C. to about 38° C., about 34° C. to about 36° C., about 35° C. to about 39° C., about 35° C. to about 38° C., about 35° C. to about 37° C., about 36° C. to about 39° C., about 36° C. to about 38° C., or about 37° C. to about 39° C.).

In some embodiments, the step of incubating the mammalian cell is performed at a temperature of about 32° C., about 33° C., about 34° C., about 35° C., about 36° C., about 37° C., about 38° C., or about 39° C.

Skilled practitioners will appreciate that the temperature can be changed at specific time point(s) in during the culturing step, e.g., on an hourly or daily basis. For example, the temperature can be changed or shifted (e.g., increased or decreased) at about one day, two days, three days, four days, five days, six days, seven days, eight days, nine days, ten days, eleven days, twelve days, fourteen days, fifteen days, sixteen days, seventeen days, eighteen days, nineteen days, or about twenty days or more after the initial seeding of the bioreactor with the cell (e.g., mammalian cell). For example, the temperature can be shifted upwards (e.g., a change of up to or about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or up to about 10 degrees Celsius). For example, the temperature can be shifted downwards (e.g., a change of up to or about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, or up to or about 20° C.).

CO₂ (Control Dissolved CO₂ to Partial Pressure in the Range of 20-200 mmHg; 80-140 mmHg)

The incubating step can include exposing the liquid medium in the bioreactor to an atmosphere containing at most or about 15% CO₂ (e.g., at most or about 14% CO₂, 12% CO₂, 10% CO₂, 8% CO₂, 6% CO₂, 5% CO₂, 4% CO₂, 3% CO₂, 2% CO₂, or at most or about 1% CO₂).

In some embodiments of any of the methods described herein, the method further includes increasing the pCO₂ by at least 10% (e.g., at least 12%, at least 14%, at least 15%, at least 16%, at least 18%, at least 20%, at least 22%, at least 24%, at least 25%, at least 26%, at least 28%, at least 30%, at least 32%, at least 34%, at least 35%, at least 36%, at least 38%, at least 40%, at least 42%, at least 44%, at least 45%, at least 46%, at least 48%, at least 50%, at least 52%, at least 54%, at least 55%, at least 56%, at least 58%, at least 60%, at least 62%, at least 64%, at least 65%, at least 66%, at least 68%, at least 70%, at least 72%, at least 74%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, or at least 99%) and increasing the pH of the first and the second liquid culture medium by at least 8% (e.g., at least 10%, at least 12%, at least 14%, at least 15%, at least 16%, at least 18%, at least 20%, at least 22%, at least 24%, at least 25%, at least 26%, at least 28%, at least 30%, at least 32%) in the vessel (e.g., any of the vessels described herein (e.g., a bioreactor)) at some point during the period of time.

In some embodiments of any of the methods described herein, the pCO₂ and the pH of the first, second, and/or third liquid culture medium in the vessel (e.g., any of the vessels described herein (e.g., a bioreactor)) are increased (e.g., at least 12%, at least 14%, at least 15%, at least 16%, at least 18%, at least 20%, at least 22%, at least 24%, at least 25%, at least 26%, at least 28%, at least 30%, at least 32%, at least 34%, at least 35%, at least 36%, at least 38%, at least 40%, at least 42%, at least 44%, at least 45%, at least 46%, at least 48%, at least 50%, at least 52%, at least 54%, at least 55%, at least 56%, at least 58%, at least 60%, at least 62%, at least 64%, at least 65%, at least 66%, at least 68%, at least 70%, at least 72%, at least 74%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, or at least 99%) based on the viable cell density over the period of time.

### Recombinant Proteins

Non-limiting examples of recombinant proteins that can be produced by the methods provided herein include immunoglobulins (including light and heavy chain immunoglobulins, antibodies, or antibody fragments (e.g., any of the antibody fragments described herein), enzymes (e.g., a galactosidase (e.g., an alpha-galactosidase), Myozyme, or Cerezyme), proteins (e.g., human erythropoietin, tumor necrosis factor (TNF), or an interferon alpha or beta), or immunogenic or antigenic proteins or protein fragments (e.g., proteins for use in a vaccine). The recombinant protein can be an engineered antigen-binding polypeptide that contains at least one multifunctional recombinant protein scaffold (see, e.g., the recombinant antigen-binding proteins described in Gebauer et al., Current Opin. Chem. Biol. 13:245-255, 2009; and U.S. Patent Application Publication No. 2012/0164066 (herein incorporated by reference in its entirety)).

Non-limiting examples of recombinant proteins that are antibodies include: panitumumab, omalizumab, abagovomab, abciximab, actoxumab, adalimumab, adecatumumab, afelimomab, afutuzumab, alacizumab, alacizumab, alemtuzumab, alirocumab, altumomab, amatuximab, amatuximab, anatumomab, anrukinzumab, apolizumab, arcitumomab, atinumab, tocilizumab, basilizimab, bectumomab, belimumab, bevacizumab, besilesomab, bezlotoxumab, biciromab, canakinumab, certolizumab, cetuximab, cixutumumab, daclizumab, denosumab, densumab, eculizumab, edrecolomab, efalizumab, efungumab, epratuzumab, ertumaxomab, etaracizumab, figitumumab, golimumab, ibritumomab tiuxetan, igovomab, imgatuzumab, infliximab, inolimomab, inotuzumab, labetuzumab, lebrikizumab, moxetumomab, natalizumab, obinutuzumab, oregovomab, palivizumab, panitumumab, pertuzumab, ranibizumab, rituximab, tocilizumab, tositumomab, tralokinumab, tucotuzumab, trastuzumab, veltuzumab, zalutumumab, and zatuximab.

Additional examples of recombinant antibodies that can be produced by the methods described herein are known in the art. Additional non-limiting examples of recombinant proteins that can be produced by the present methods include: alglucosidase alfa, laronidase, abatacept, galsulfase, lutropin alfa, antihemophilic factor, agalsidase beta, interferon beta-1a, darbepoetin alfa, tenecteplase, etanercept, coagulation factor IX, follicle stimulating hormone, interferon beta-1a, imiglucerase, dornase alfa, epoetin alfa, insulin or insulin analogs, mecasermin, factor VIII, factor VIIa, anti-thrombin III, protein C, human albumin, erythropoietin, granulocyte colony stimulating factor, granulocyte macrophage colony stimulating factor, interleukin-11, laronidase, idursuphase, galsulphase, α-1-proteinase inhibitor, lactase, adenosine deaminase, tissue plasminogen activator, thyrotropin alpha (e.g., Thyrogen®) and alteplase. Additional examples of recombinant proteins that can be produced by the present methods include acid α-glucosidase, alglucosidase alpha (e.g., Myozyme® and Lumizyme®), α-L-iduronidase (e.g., Aldurazyme®), iduronate sulfatase, heparan N-sulfatase, galactose-6-sulfatase, acid β-galactosidase, β-glucoronidase, N-acetylglucosamine-1-phosphotransferase, α-N-acetylgalactosaminidase, acid lipase, lysosomal acid ceramidase, acid sphingomyelinase, β-glucosidase (e.g., Cerezyme® and Ceredase®), galactosylceramidase, α-galactosidase-A (e.g., Fabrazyme®), acid β-galactosidase, β-galactosidase, neuraminidase, hexosaminidase A, and hexosaminidase B.

A secreted, soluble recombinant protein can be recovered from the liquid culture medium (e.g., one or more of the first, second, and third liquid culture medium) by removing or otherwise physically separating the liquid medium from the cells (e.g., mammalian cells). A variety of different methods for removing liquid culture medium from cells (e.g., mammalian cells) are known in the art, including, for example, centrifugation, filtration, pipetting, and/or aspiration. The secreted recombinant protein can then be recovered and further purified from the liquid culture medium using a variety of biochemical techniques including various types of chromatography (e.g., affinity chromatography, molecular sieve chromatography, cation exchange chromatography, or anion exchange chromatography) and/or filtration (e.g., molecular weight cut-off filtration).

### Recovering the Recombinant Protein

Some embodiments of the methods described herein further include recovering the recombinant protein (e.g., any of the recombinant proteins described herein) from the mammalian cell and/or one or more of the first, second, and third liquid culture medium. In some examples, the recovering includes lysing the mammalian cells. In other examples, recovering can include collecting the recombinant protein from the medium (e.g., one or more of the first, second, and third liquid culture medium).

Collecting can be performed after the culturing achieves a viable cell density of, e.g., greater than about 60×10⁶ cells/mL, 62×10⁶ cells/mL, 64×10⁶ cells/mL, 66×10⁶ cells/mL, 68×10⁶ cells/mL, 70×10⁶ cells/mL, 72×10⁶ cells/mL, 74×10⁶ cells/mL, 76×10⁶ cells/mL, 78×10⁶ cells/mL, 80×10⁶ cells/mL, greater than about 82×10⁶ cells/mL, greater than about 84×10⁶ cells/mL, greater than about 86×10⁶ cells/mL, greater than about 88×10⁶ cells/mL, greater than about 90×10⁶ cells/mL, greater than about 92×10⁶ cells/mL, greater than about 94×10⁶ cells/mL, greater than about 96×10⁶ cells/mL, greater than about 98×10⁶ cells/mL, greater than about 100×10⁶ cells/mL, greater than about 102×10⁶ cells/mL, greater than about 104×10⁶ cells/mL, greater than about 106×10⁶ cells/mL, greater than about 108×10⁶ cells/mL, greater than about 110×10⁶ cells/mL, greater than about 112×10⁶ cells/mL, greater than about 114×10⁶ cells/mL, greater than about 116×10⁶ cells/mL, greater than about 118×10⁶ cells/mL, greater than about 120×10⁶ cells/mL, greater than about 122×10⁶ cells/mL, greater than about 124×10⁶ cells/mL, greater than about 126×10⁶ cells/mL, greater than about 128×10⁶ cells/mL, greater than about 130×10⁶ cells/mL, greater than about 132×10⁶ cells/mL, greater than about 134×10⁶ cells/mL, greater than about 136×10⁶ cells/mL, greater than about 138×10⁶ cells/mL, greater than about 140×10⁶ cells/mL, greater than about 142×10⁶ cells/mL, greater than about 144×10⁶ cells/mL, greater than about 146×10⁶ cells/mL, greater than about 148×10⁶ cells/mL, greater than about 150×10⁶ cells/mL, greater than about 152×10⁶ cells/mL, greater than about 154×10⁶ cells/mL, greater than about 156×10⁶ cells/mL, greater than about 158×10⁶ cells/mL, greater than about 160×10⁶ cells/mL, greater than about 162×10⁶ cells/mL, greater than about 164×10⁶ cells/mL, greater than about 166×10⁶ cells/mL, greater than about 168×10⁶ cells/mL, greater than about 170×10⁶ cells/mL, greater than about 172×10⁶ cells/mL, greater than about 174×10⁶ cells/mL, greater than about 176×10⁶ cells/mL, greater than about 178×10⁶ cells/mL, greater than about 180×10⁶ cells/mL, greater than about 182×10⁶ cells/mL, greater than about 184×10⁶ cells/mL, greater than about 186×10⁶ cells/mL, greater than about 188×10⁶ cells/mL, greater than about 190×10⁶ cells/mL, greater than about 192×10⁶ cells/mL, greater than about 194×10⁶ cells/mL, greater than about 196×10⁶ cells/mL, greater than about 198×10⁶ cells/mL, or greater than about 200×10⁶ cells/mL).

### Formulating the Recombinant Protein

Some embodiments of any of the methods described herein further include a step of formulating the recombinant protein (e.g., a recombinant protein produced by any of the methods described herein, e.g., a recovered recombinant protein or a recovered and purified recombinant protein) into a pharmaceutical composition. For example, formulating can include adding a pharmaceutically acceptable excipient to the recombinant protein (e.g., a recombinant protein produced by any of the methods described herein, e.g., a recovered recombinant protein or a recovered and purified recombinant protein). Formulating can include mixing a pharmaceutically acceptable excipient with the recombinant protein (e.g., a recombinant protein produced by any of the methods described herein, e.g., a recovered recombinant protein or a recovered and purified recombinant protein). Examples of pharmaceutically acceptable excipients (e.g., non-naturally occurring pharmaceutically acceptable excipients) are well known in the art. In some embodiments, the recombinant protein (e.g., a recombinant protein produced by any of the methods described herein, e.g., a recovered recombinant protein or a recovered and purified recombinant protein) is formulated for intravenous, intraarterial, subcutaneous, intraperitoneal, or intramuscular administration.

### Pharmaceutical Compositions and Kits

Also provided herein are pharmaceutical compositions that include at least one of any of the recombinant proteins described herein (e.g., produced by any of the methods described herein). The pharmaceutical compositions can be formulated in any manner known in the art. The pharmaceutical compositions are formulated to be compatible with their intended route of administration (e.g., intravenous, intraarterial, subcutaneous, intraperitoneal, or intramuscular administration).

In some embodiments, the pharmaceutical compositions can include a pharmaceutically acceptable carrier (e.g., phosphate buffered saline). Single or multiple administrations of formulations can be given depending on, for example, the dosage and frequency as required and tolerated by the subject.

Also provided herein are kits that include a pharmaceutical compositions (e.g., any of the pharmaceutical compositions described herein). In some embodiments, the kits include instructions for performing any of the methods described herein. In some embodiments, the kits can include at least one dose (e.g., 2, 3, 4, 5, 6, 7, 8, 9 or 10) doses of any of the pharmaceutical compositions described herein.

### Methods of Treating

Provided herein are treating a subject in need thereof that include: administering a therapeutically effective amount of any of the pharmaceutical compositions described herein that include at least one of any of the recombinant proteins described herein (produced by any of the methods described herein), to the subject.

Administering can be performed, e.g., at least once (e.g., at least 2-times, at least 3-times, at least 4-times, at least 5-times, at least 6-times, at least 7-times, at least 8-times, at least 9-times, at least 10-times, at least 11-times, or at least 12-times) a week.

This invention is further described in the following examples, which do not limit the scope of the invention described in the claims.

### EXAMPLES

### Example 1. Methods of Osmolality Control

Methods involving perfusion culturing use a lot of liquid culture medium and require significant costs for preparation of each batch. Most of the liquid culture medium is water. However, a single liquid culture medium cannot be used for the entirety of a high cell density perfusion process because at high cell densities, there is a significant drop in osmolality due to nutrient consumption. For example, 12 g/L glucose consumption results in a 67 mOsm/kg decrease.

Use of a single liquid culture medium with a typical osmolality (290 mOsm/kg to 320 mOsm/kg) resulted in low osmolality at high cell density. Hypoosmotic conditions resulted in poor cell health and reduced productivity. However, use of a single high osmolality liquid culture medium from the beginning of a cell culture (e.g., a perfusion cell culture) may result in runaway lactate production and cell culture decline.

The second liquid culture medium (4× liquid culture medium formulation) described herein (added to a first liquid culture medium) can have a relatively high osmolality (relative to a feed liquid culture medium for fed-batch culturing), and can have a pH of less than 7 to prevent precipitation. This second liquid culture medium can be stable for less than 1 week at room temperature even at pH 6 to pH 7. At least two alternatives can be used to increase second liquid culture medium stability: 1) remove cysteine and tyrosine, or 2) use alternative forms of cysteine and tyrosine (e.g., pyruvate/cysteine and glycine-tyrosine, respectively).

In order to control osmolality in a perfusion cell culture, a concentrated second liquid culture medium can be used and can be diluted with different amounts of a third low osmolality liquid (e.g. water). A concentrated sodium chloride (NaCl) solution can also, optionally, be added if an increase in osmolality is required. In addition, dissolved CO₂ concentration and pH of the culture can be varied to modulate osmolality.

### Example 2. Impact of Osmolality on Cell Culture Performance

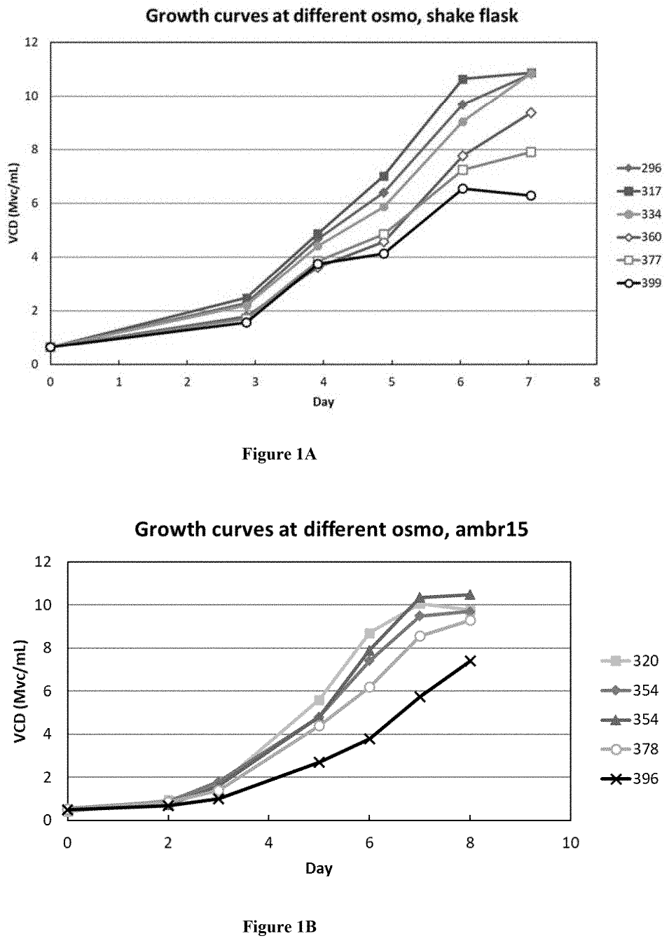

To determine the impact of osmolality on cell culture performance (e.g., viable cell density), mammalian cells were grown in a liquid culture medium having an osmolality between about 290 mOsm/kg and about 400 mOsm/kg. FIGS. 1A and 1B show the viable cell density over time in mammalian cells expressing Protein 1 and Protein 2, respectively. The data of FIGS. 1A and 1B show that slower growth of mammalian cells was observed when mammalian cells were grown in a liquid culture medium having an osmolality of greater than 380 mOsm/kg.

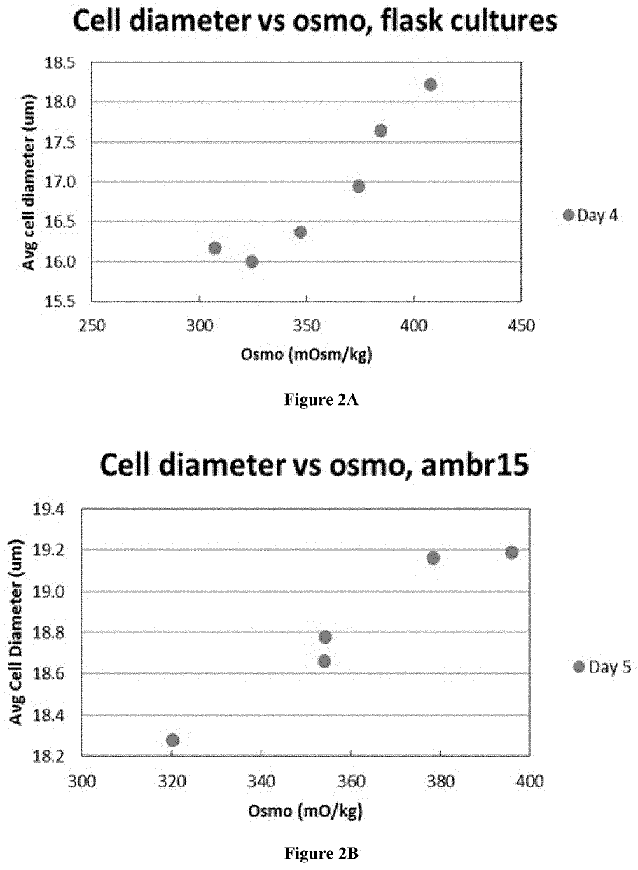

On days 4 and 5, respectively, the average cell diameter was shown at the tested osmolalities (FIGS. 2A and 2B). The data of FIGS. 2A and 2B showed larger average cell size at higher osmolality, illustrating that osmolality impacts cell morphology.

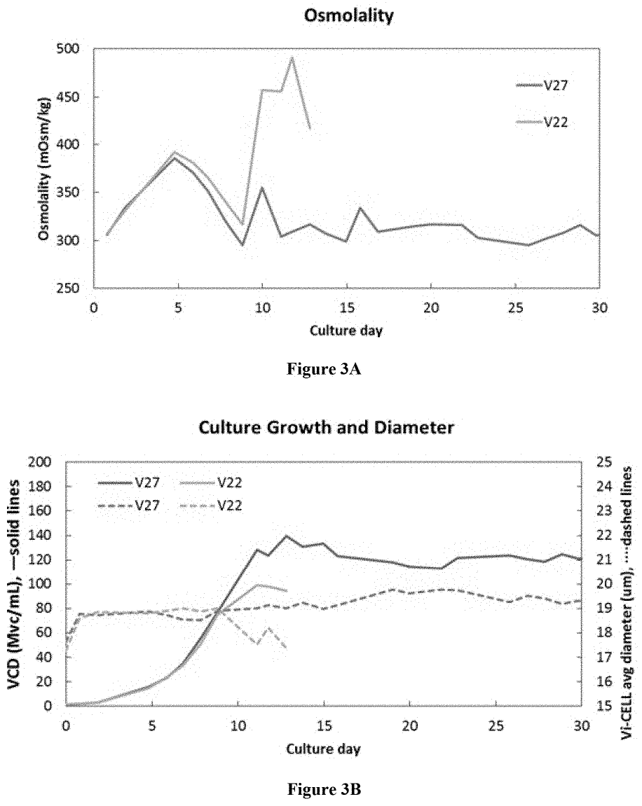

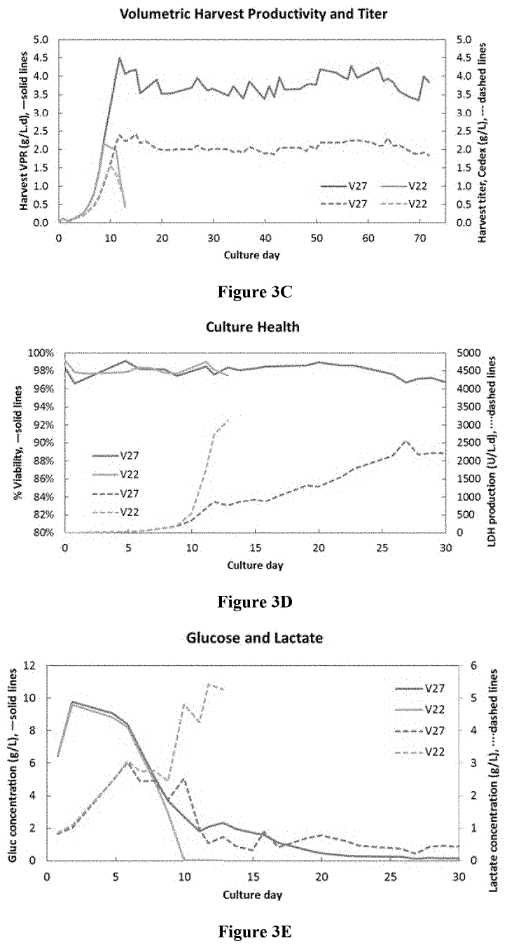

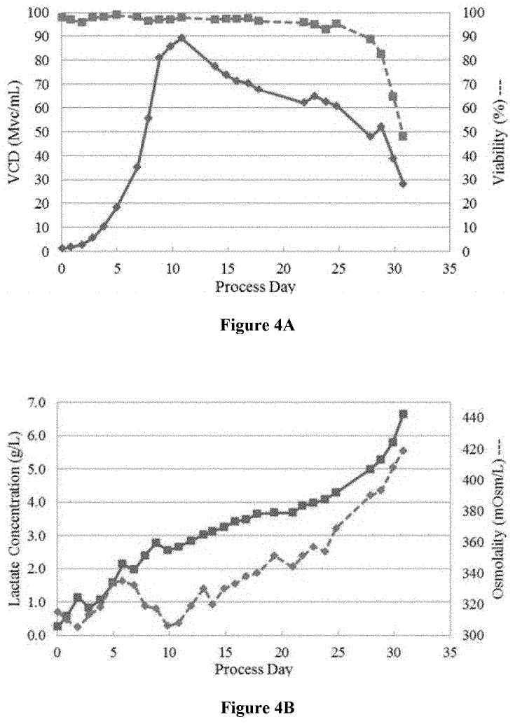

FIGS. 3A and 3B show osmolality and cell culture growth of two bioreactors being fed concentrated cell culture medium (osmolality of 1320 mOsm/kg) and water, respectively. The ratio of concentrated medium feed to water feed was varied in the growth phase of the culture. Vessel 22 (“V22”) had a lower ratio of water to medium than Vessel 27 (“V27”), resulting in high osmolality in the culture beginning on Day 10 (FIG. 3A). This resulted in a reduced growth rate of V22 relative to V27 (FIG. 3B). The high osmolality also resulted in decreased productivity (FIG. 3C) and higher lactate dehydrogenase production, indicating decreased culture health (FIG. 3D). One response to high osmolality is increased lactate production in V22 (FIG. 3E), which will then continue to drive lactate higher (“runaway lactate production”). FIGS. 4A and 4B show a culture with high lactate production, resulting in increased osmoalality. Once osmolality reached>400 mOsm/kg, the rate of lactate production increased and cell growth and viability declined.

### Example 3. Options for Osmolality Control

Osmolality threshold for perfusion cultures for safe operation was in the range of 360-420 mOsm/kg at the high end. Osmolality can be controlled through various means, for example, 1) by feeding concentrated cell culture medium and modulating water dilution rate in conjunction with feedback control, 2) vary process parameters to influence base addition, e.g., high pH and pCO₂ when higher osmolality is required, 3) using at least two different liquid culture media within a bioreactor campaign, e.g., growth liquid culture media with low osmolality and production liquid culture media with high osmolality, 4) add salt at high cell density to increase osmolality, e.g., NaCl and/or KCl with feedback control. Process changes to control osmolality can be performed manually based on offline osmolality measurements of the cell-free culture permeate. Alternatively, automated feedback control can be implemented based on online osmolality predictions. Online osmolality can be predicted from online capacitance measurements, online conductivity measurements, or Raman spectroscopy measurements in conjunction with appropriate models.

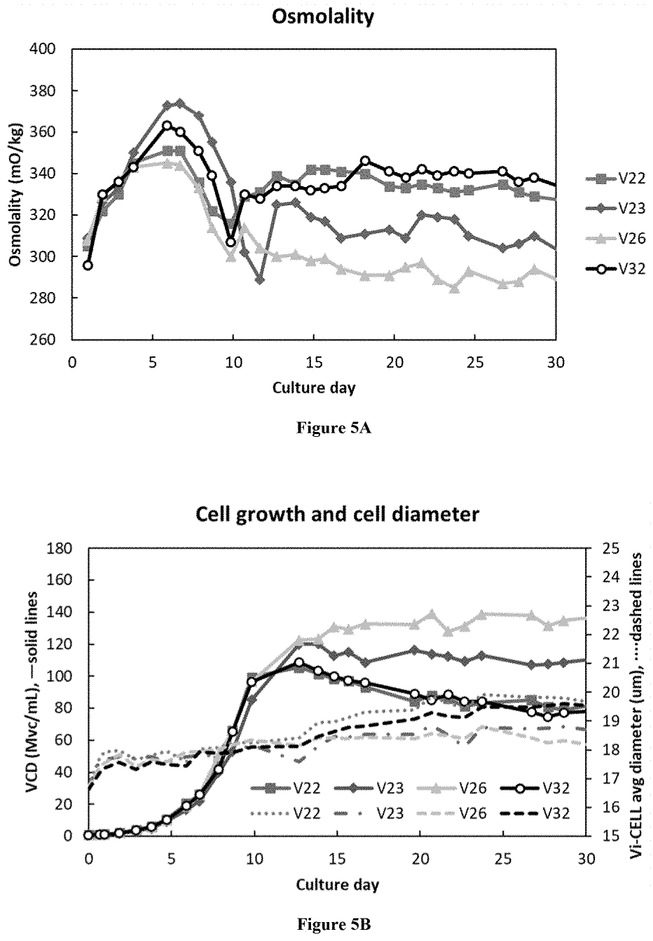

FIGS. 5A and 5B showed a step change in feed medium osmolality going from growth to production phase. For each bioreactor, NaCl addition was manually turned on at given time point, then the NaCl flow was kept constant for the duration of the experiment. The data of FIGS. 5A and 5B showed different VCD profiles at different osmolalities, despite the same capacitance set point. This illustrates a limitation of using a constant NaCl feed rate to control osmolality.

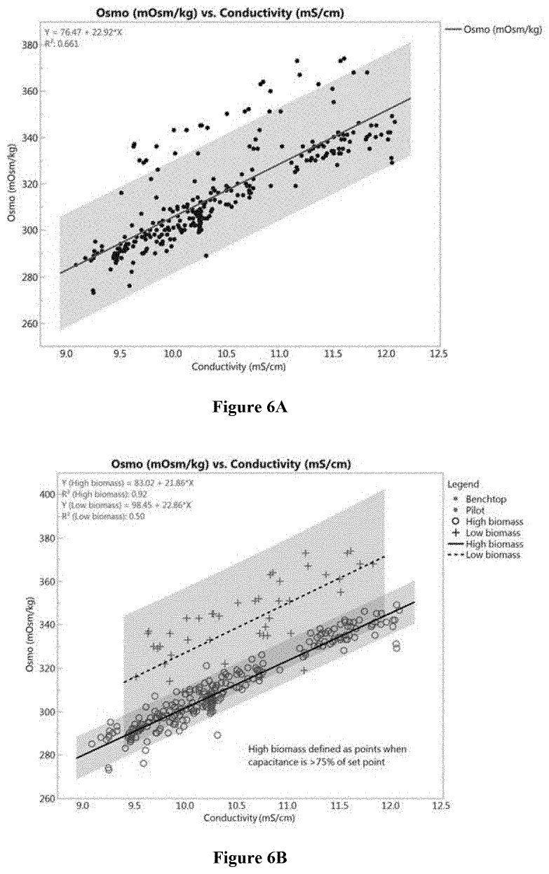

FIGS. 6A and 6B showed good correlation of osmolality and conductivity at high cell density during the harvest phase when osmolality control was needed.

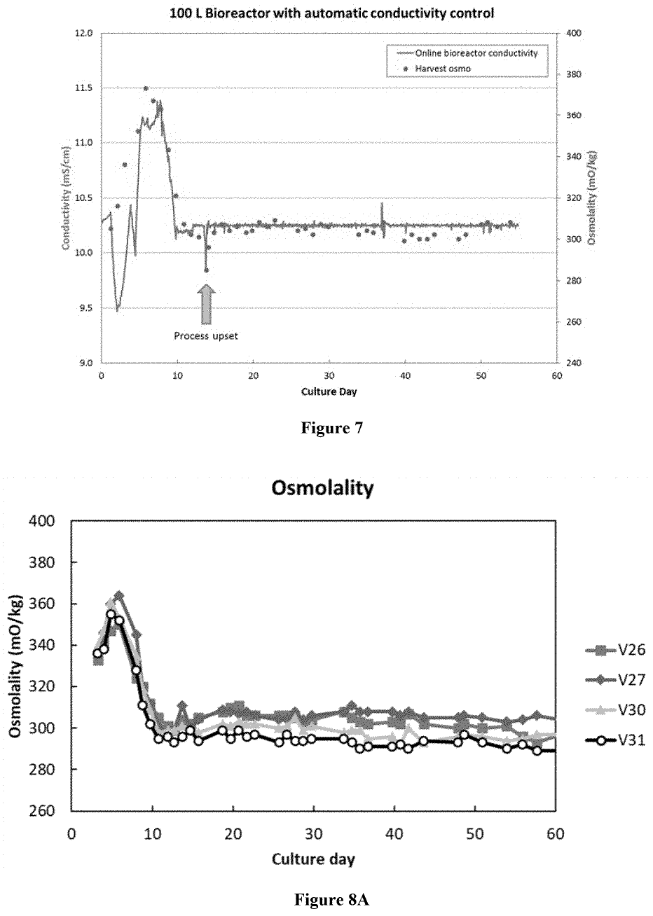

Conductivity was measured by ABER probes and the average of two measurements was plotted in FIG. 7. To maintain the set point, 5 M NaCl was fed to the bioreactor based on conductivity feedback control. As shown in FIG. 7, control was started on day 5, and no salt addition was needed earlier in the culturing period. NaCl addition began around Day 10. NaCl addition based on conductivity feedback control resulted in consistent conductivity and osmolality throughout the harvest phase of the culture. Additionally, NaCl addition based on conductivity feedback control helps reduce osmolality excursions during process upsets relative to a process using constant NaCl addition.

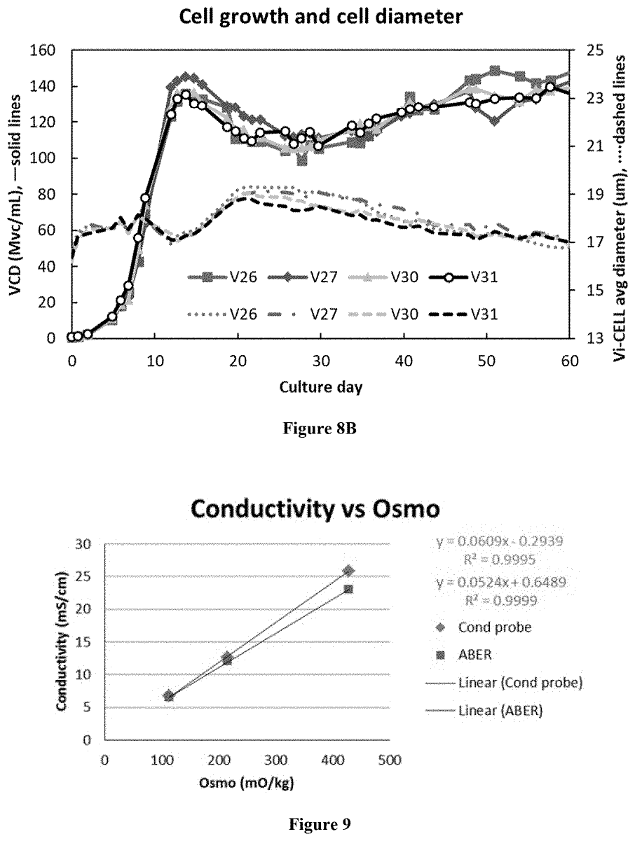

The data in FIGS. 8A and 8B showed similar viable cell density profiles at the same capacitance set point for all four experimental runs in which automated conductivity control produced similar osmolality trends.

FIG. 9 showed good correlation between conductivity and osmolality with two different probe types.

FIG. 10 showed osmolality can be accurately predicted with Raman spectroscopy, and the Raman probe can be placed in a bioreactor or in cell-free perfusion harvest.

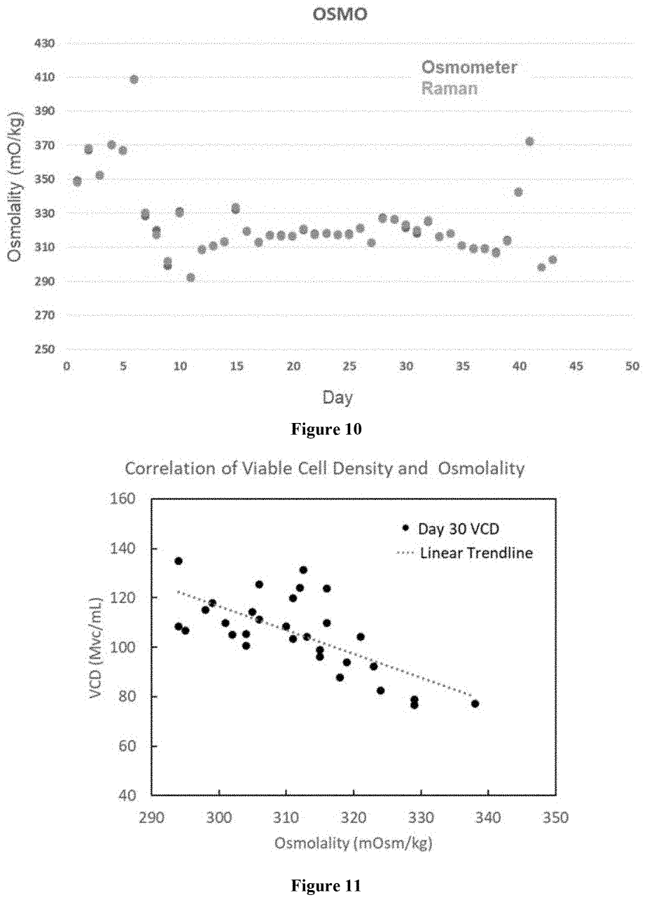

As shown in FIG. 11, a trend was observed in which viable cell density in a perfusion cell culture process reached a low point midway through the process. This occurred due to cell size increase when the biomass concentration was controlled to maintain a constant capacitance value, as measured with a capacitance sensor in the bioreactor. In the latter half of these cultured, cell size decreased and viable cell density would rise.

When data from multiple batches was compiled, it was discovered that there was a correlation between osmolality and the lowest viable cell density reached in the cultures, with higher osmolality correlating with lower cell density. This finding is illustrated in the figure, in which VCD on Day 30 of a 60-day process is plotted versus the culture osmolality on Day 30. With the ability to control osmolality at a desired level, changes in VCD during the process could be minimized and intra-process consistency was improved.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of perfusion culturing a mammalian cell, the method comprising:
providing a vessel containing a mammalian cell disposed in a first liquid culture medium having an osmolality of about 270 mOsm/kg to about 380 mOsm/kg;
incubating the mammalian cell for a period of time at about 32° C. to about 39° C.; and
during the period of time, continuously or periodically removing a first volume of the first liquid culture medium and adding to the first liquid culture medium a second volume of a second liquid culture medium, wherein the first and second volumes are about equal and the osmolality of the first and second liquid culture medium in the vessel is maintained at about 270 mOsm/kg to about 380 mOsm/kg over the period of time.

2. The method of claim 1, wherein the first liquid culture medium has an osmolality of about 270 mOsm/kg to about 320 mOsm/kg.

3. The method of claim 1, wherein the second liquid culture medium comprises greater than 8 g/L of a poloxamer.

4.-5. (canceled)

6. The method of claim 3, wherein the poloxamer is Pluronic F68.

7. (canceled)

8. The method of claim 1, wherein the second liquid culture medium comprises sodium chloride.

9. The method of claim 1, wherein the second liquid culture medium has an osmolality of about 800 mOsm/kg to about 2,500 mOsm/kg.

10.-12. (canceled)

13. The method of claim 1, wherein maintaining the osmolality of the first and second liquid culture medium in the vessel comprises adjusting the flow rate of the second liquid culture medium at some point during the period of time.

14. The method of claim 13, wherein the adjusting step comprises increasing the flow rate of the second liquid culture medium at some point during the period of time.

15. The method of claim 14, wherein the method further comprises adjusting one or both of the pH and the pCO₂ of the first and second liquid culture medium in the vessel at some point during the period of time.

16. The method of claim 15, wherein the adjusting step comprises decreasing the flow rate of the second liquid culture medium at some point during the period of time.

17. The method of claim 1, wherein the method further comprises increasing the pCO₂ and increasing the pH of the first and the second liquid culture medium in the vessel over the period of time.

18. The method of claim 17, wherein the pCO₂ and the pH of the first and the second liquid culture medium in the vessel are increased based on the viable cell density over the period of time.

19.-21. (canceled)

22. The method of claim 1, wherein the method further comprises monitoring the osmolality of the first and second liquid culture medium in the vessel during the period of time.

23.-25. (canceled)

26. The method of claim 1, wherein the method further comprises periodically adding a third volume of an aqueous solution to the first and the second liquid culture medium in the vessel during the period of time, wherein the first volume and the sum of the second and third volumes are about equal and the osmolality of the first, second, and third liquid culture medium in the vessel is maintained at about 270 mOsm/kg to about 380 mOsm/kg over the period of time.

27.-31. (canceled)

32. The method of claim 1, wherein the method further includes continuously or periodically adding to the first liquid culture medium a third volume of a third liquid culture medium, wherein the first volume and the sum of the second and third volumes are about equal and the osmolality of the first, second, and third liquid culture medium in the vessel is maintained at about 270 mOsm/mg to about 380 mOsm/kg over the period of time.

33.-52. (canceled)

53. The method of claim 1, wherein the vessel is a bioreactor.

54. (canceled)

55. A method of producing a recombinant protein, the method comprising:
providing a vessel containing a mammalian cell containing a nucleic acid encoding a recombinant protein disposed in a first liquid culture medium having an osmolality of about 270 mOsm/kg to about 380 mOsm/kg;
incubating the mammalian cell for a period of time at about 32° C. to about 39° C.; and
during the period of time, continuously or periodically removing a first volume of the first liquid culture medium and adding to the first liquid culture medium a second volume of a second liquid culture medium, wherein the first and second volumes are about equal and the osmolality of the first and second liquid culture medium in the vessel is maintained at about 270 mOsm/kg to about 380 mOsm/kg over the period of time; and
recovering the recombinant protein from the mammalian cell or from the first and/or second liquid culture medium.

56. The method of claim 55, wherein the first liquid culture medium has an osmolality of about 270 mOsm/kg to about 320 mOsm/kg.

57.-62. (canceled)

63. The method of claim 55, wherein the second liquid culture medium has an osmolality of about 800 mOsm/kg to about 2,500 mOsm/kg.

64.-114. (canceled)

115. The method of claim 55, wherein the method further comprises isolating the recombinant protein.

116. The method of claim 115, wherein the method further comprises formulating the isolated recombinant protein.

117. (canceled)

118. A pharmaceutical composition including a recombinant protein of claim 117.

119. A kit comprising a pharmaceutical composition of claim 118.

120. A method of treating a subject in need thereof comprising administering a therapeutically effective amount of a pharmaceutical composition of claim 118 to the subject.
